(19) United States
(12) Patent Application Publication
(10) Pub. No.: US 2023/0255479 A1
(43) Pub. Date: Aug. 17, 2023

(54) SYSTEM AND METHOD FOR EYE REGION VISUALIZATION IN A LASER SURGICAL SYSTEM

(71) Applicant: ViaLase, Inc., Aliso Viejo, CA (US)

(72) Inventors: Guy Holland, San Juan Capistrano, CA (US); Tibor Juhasz, San Clemente, CA (US); Wesley W. Lummis, Rancho Santa Margarita, CA (US); Eric R. Mikula, Aliso Viejo, CA (US); Ferenc Raksi, Mission Viejo, CA (US); Manu Sharma, Ladera Ranch, CA (US); Hadi Srass, Yorba Linda, CA (US); Carlos G. Suarez, Tustin, CA (US)

(21) Appl. No.: 17/671,496

(22) Filed: Feb. 14, 2022

(51) Int. Cl. A61B 3/14; H04N 5/232

(52) U.S. Cl. CPC A61B 3/14; H04N 5/232125; H04N 5/23299; H04N 5/2256

(57) ABSTRACT

A system for imaging a region of an eye includes a laser source that outputs a laser beam; a first optical subsystem that couples to the eye; a focusing objective coupled with the first optical subsystem and the laser source; a visualization system having a depth of field and a field of view; a movement subsystem that moves the focusing objective and the visualization system; and a control system that controls the movement subsystem and the visualization system to: place the depth of field and the field of view at respective positions relative to a target volume so the volume is within the depth of field and the field of view, obtain an image of the region of the eye, and maintain the position of the field of view of the visualization system relative to the target volume during movement of a laser focus through the target volume.

### TECHNICAL FIELD

The present disclosure relates generally to the field of medical devices and treatment of diseases in ophthalmology including glaucoma, and more particularly to systems and methods for visualization of a region of the eye in a laser surgical system.

### BACKGROUND

Before describing the different types of glaucoma and current diagnosis and treatments options, a brief overview of the anatomy of the eye is provided.

Anatomy of the Eye

With reference to FIGS. 1-3, the outer tissue layer of the eye 1 includes a sclera 2 that provides the structure of the eye's shape. In front of the sclera 2 is a cornea 3 that is comprised of transparent layers of tissue that allow light to enter the interior of the eye. Inside the eye 1 is a crystalline lens 4 that is connected to the eye by fiber zonules 5, which are connected to the ciliary body 6. Between the crystalline lens 4 and the cornea 3 is an anterior chamber 7 that contains a flowing clear liquid called aqueous humor 8. Encircling the perimeter of the crystalline lens 4 is an iris 9 which forms a pupil around the approximate center of the crystalline lens. As shown in FIG. 2, a posterior chamber 23 is an annular volume behind the iris 9 and bounded by the ciliary body 6, fiber zonules 5, and the crystalline lens 4. The vitreous humor 10 is located between the crystalline lens 4 and the retina 11. Light entering the eye is optically focused through the cornea 3 and crystalline lens.

With reference to FIG. 2, the corneoscleral junction of the eye is the portion of the anterior chamber 7 at the intersection of the iris 9, the sclera 2, and the cornea 3. The anatomy of the eye 1 at the corneoscleral junction includes a trabecular meshwork 12. The trabecular meshwork 12 is a fibrous network of tissue that encircles the iris 9 within the eye 1. In simplified, general terms the tissues of the corneoscleral junction are arranged as follows: the iris 9 meets the ciliary body 6, the ciliary body meets with the underside of the scleral spur 14, the top of the scleral spur serves as an attachment point for the bottom of the trabecular meshwork 12. The ciliary body is present mainly in the posterior chamber, but also extends into the very corner of the anterior chamber 7. The network of tissue layers that make up the trabecular meshwork 12 are porous and thus present a pathway for the egress of aqueous humor 8 flowing from the anterior chamber 7. This pathway may be referred to herein as an aqueous humor outflow pathway, an aqueous outflow pathway, or simply an outflow pathway.

Referring to FIG. 3, the pathway formed by the pores in the trabecular meshwork 12 connect to a set of thin, porous tissue layers called the uveal 15, the corneoscleral meshwork 16, and the juxtacanalicular tissue 17. The juxtacanalicular tissue 17, in turn, abuts a structure called Schlemm's canal 18. The Schlemm's canal 18 carries a mixture of aqueous humor 8 and blood from the surrounding tissue to drain into the venous system though a system of collector channels 19. As shown in FIG. 2, the vascular layer of the eye, referred to as the choroid 20, is next to the sclera 2. A space, called the suprachoroidal space 21, may be present between the choroid 20 and the sclera 2. The general region near the periphery of the wedge between the cornea 3 and the iris 9, running circumferentially is called the irido-corneal angle 13. The irido-corneal angle 13 may also be referred to as the corneal angle of the eye or simply the angle of the eye. The ocular tissues illustrated in FIG. 3 are all considered to be within the irido-corneal angle 13.

With reference to FIG. 4, two possible outflow pathways for the movement of aqueous humor 8 include a trabecular outflow pathway 40 and a uveoscleral outflow pathway 42. With additional reference to FIG. 2, aqueous humor 8, which is produced by the ciliary body 6, flows from the posterior chamber 23 through the pupil into the anterior chamber 7, and then exits the eye through one or more of the two different outflow pathways 40, 42. Approximately 90% of the aqueous humor 8 leaves via the trabecular outflow pathway 40 by passing through the trabecular meshwork 12, into the Schlemm's canal 18 and through one or more plexus of collector channels 19 before draining through a drain path 41 into the venous system. Any remaining aqueous humor 8 leaves primarily through the uveoscleral outflow pathway 42. The uveoscleral outflow pathway 42 passes through the ciliary body 6 face and iris root into the suprachoroidal space 21 (shown in FIG. 2). Aqueous humor 8 drains from the suprachoroidal space 21, from which it can be drained through the sclera 2.

The intra-ocular pressure of the eye depends on the aqueous humor 8 outflow through the trabecular outflow pathway 40 and the resistance to outflow of aqueous humor through the trabecular outflow pathway. The intra-ocular pressure of the eye is largely independent of the aqueous humor 8 outflow through the uveoscleral outflow pathway 42. Resistance to the outflow of aqueous humor 8 through the trabecular outflow pathway 40 may lead to elevated intra-ocular pressure of the eye, which is a widely recognized risk factor for glaucoma. Resistance through the trabecular outflow pathway 40 may increase due to a collapsed or malfunctioning Schlemm's canal 18 and trabecular meshwork 12.

Referring to FIG. 5, as an optical system, the eye 1 is represented by an optical model described by idealized centered and rotationally symmetrical surfaces, entrance and exit pupils, and six cardinal points: object and image space focal points, first and second principal planes, and first and second nodal points. Angular directions relative to the human eye are often defined with respect to an optical axis 24, a visual axis 26, a pupillary axis 28 and a line of sight 29 of the eye. The optical axis 24 is the symmetry axis, the line connecting the vertices of the idealized surfaces of the eye. The visual axis 26 connects the foveal center 22 with the first and second nodal points to the object. The line of sight 29 connects the fovea through the exit and entrance pupils to the object. The pupillary axis 28 is normal to the anterior surface of the cornea 3 and is directed to the center of the entrance pupil. These axes of the eye differ from one another only by a few degrees and fall within a range of what is generally referred to as the direction of view.

Glaucoma

Glaucoma is a group of diseases that can harm the optic nerve and cause vision loss or blindness. It is the leading cause of irreversible blindness. Approximately 80 million people are estimated to have glaucoma worldwide and of these, approximately 6.7 million are bilaterally blind. More than 2.7 million Americans over age 40 have glaucoma. Symptoms start with loss of peripheral vision and can progress to blindness.

There are two forms of glaucoma, one is referred to as closed-angle glaucoma, the other as open-angled glaucoma. With reference to FIGS. 1-4, in closed-angle glaucoma, the iris 9 in a collapsed anterior chamber 7 may obstruct and close off the flow of aqueous humor 8. In open-angle glaucoma, which is the more common form of glaucoma, the permeability of ocular tissue may be affected by irregularities in the juxtacanalicular tissue 17 and inner wall of Schlemm's canal 18a, and blockage of tissue in the irido-corneal angle 13 along the trabecular outflow pathway 40.

As previously stated, elevated intra-ocular pressure (TOP) of the eye, which damages the optic nerve, is a widely recognized risk factor for glaucoma. However, not every person with increased eye pressure will develop glaucoma, and glaucoma can develop without increased eye pressure. Nonetheless, it is desirable to reduce elevated TOP of the eye to reduce the risk of glaucoma.

Methods of diagnosing conditions of the eye of a patient with glaucoma include visual acuity tests and visual field tests, dilated eye exams, tonometry, i.e., measuring the intra-ocular pressure of the eye, and pachymetry, i.e., measuring the thickness of the cornea. Deterioration of vision starts with the narrowing of the visual field and progresses to total blindness. Imaging methods include slit lamp examination, observation of the irido-corneal angle with a gonioscopic lens and optical coherence tomography (OCT) imaging of the anterior chamber and the retina.

Once diagnosed, some clinically proven treatments are available to control or lower the intra-ocular pressure of the eye to slow or stop the progress of glaucoma. The most common treatments include: 1) medications, such as eye drops or pills, 2) laser surgery, and 3) traditional surgery. Treatment usually begins with medication. However, the efficacy of medication is often hindered by patient non-compliance. When medication does not work for a patient, laser surgery is typically the next treatment to be tried. Traditional surgery is invasive, more high risk than medication and laser surgery, and has a limited time window of effectiveness. Traditional surgery is thus usually reserved as a last option for patients whose eye pressure cannot be controlled with medication or laser surgery.

Laser Surgery

With reference to FIG. 2, laser surgery for glaucoma targets the trabecular meshwork 12 to decrease aqueous humor 8 flow resistance. Common laser treatments include Argon Laser Trabeculoplasty (ALT), Selective Laser Trabeculoplasty (SLT) and Excimer Laser Trabeculectomy (ELT).

ALT was the first laser trabeculoplasty procedure. During the procedure, an argon laser of 514 nm wavelength is applied to the trabecular meshwork 12 around 180 degrees of the circumference of the irido-corneal angle 13. The argon laser induces a thermal interaction with the ocular tissue that produces openings in the trabecular meshwork 12. ALT, however, causes scarring of the ocular tissue, followed by inflammatory responses and tissue healing that may ultimately close the opening through the trabecular meshwork 12 formed by the ALT treatment, thus reducing the efficacy of the treatment. Furthermore, because of this scarring, ALT therapy is typically not repeatable.

SLT is designed to lower the scarring effect by selectively targeting pigments in the trabecular meshwork 12 and reducing the amount of heat delivered to surrounding ocular tissue. During the procedure, a solid-state laser of 532 nm wavelength is applied to the trabecular meshwork 12 between 180 to 360 degrees around the circumference of the irido-corneal angle 13 to remove the pigmented cells lining the trabeculae which comprise the trabecular meshwork. The collagen ultrastructure of the trabecular meshwork is preserved during SLT. 12. SLT treatment can be repeated, but subsequent treatments have lower effects on TOP reduction.

ELT uses a 308 nm wavelength ultraviolet (UV) excimer laser and non-thermal interaction with ocular tissue to treat the trabecular meshwork 12 and inner wall of Schlemm's canal 18a in a manner that does not invoke a healing response. Therefore, the TOP lowering effect lasts longer. However, because the UV light of the laser cannot penetrate deep into the eye, the laser light is delivered to the trabecular meshwork 12 via an optical fiber inserted into the eye 1 through an opening and the fiber is brought into contact with the trabecular meshwork. The procedure is highly invasive and is generally practiced simultaneously with cataract procedures when the eye is already surgically open. Like ALT and SLT, ELT also lacks control over the amount of TOP reduction.

Visualization of the trabecular meshwork 12 and surrounding anatomy is a critical component of laser surgery for glaucoma. To adjust laser energy for patient anatomy or pigmentation, direct visualization of the trabecular meshwork 12 and surrounding anatomy is vital. However, in existing laser surgery systems, visualization systems for visualizing the trabecular meshwork 12 and surrounding anatomy are coupled to a laser objective that scans a laser pattern during treatment. This coupling of the visualization system to the laser objective tends to degrade the image quality of the visualization system during treatment and may adversely affect the functionality and usability of the laser surgery system.

Due to this degradation in image quality, what is needed are systems, apparatuses, and methods for laser surgery treatment of the eye that provide imaging of the trabecular meshwork 12 and surrounding anatomy during laser scanning that has a stable, consistent field of view and quality visual characteristics, e.g., focus, resolution, magnification, etc.

### SUMMARY

The present disclosure relates to a system for imaging a region of an eye having a target volume of ocular tissue. The system includes a laser source configured to output a laser beam; a first optical subsystem configured to be coupled to the eye; a focusing objective optically coupled with the first optical subsystem and the laser source; a visualization system having a depth of field and a field of view, the visualization system optically coupled to the focusing objective and to the first optical subsystem through the focusing objective; a movement subsystem configured to move the focusing objective and the visualization system; and a control system. The control system is configured to control the movement subsystem and the visualization system to: place the depth of field and the field of view of the visualization system at respective positions relative to the target volume of ocular tissue so the target volume is within the depth of field and within the field of view, obtain an image of the region of the eye with the visualization system, and maintain the position of the field of view of the visualization system relative to the target volume of ocular tissue during movement of a laser focus of the laser beam to a depth location within the target volume of ocular tissue.

The present disclosure also relates to a method of imaging a region of an eye including a target volume of ocular tissue. The method includes placing a depth of field and a field of view of a visualization system at respective positions relative to the target volume of ocular tissue and obtaining an image of the region of the eye with the visualization system. The method further includes maintaining the position of the field of view of the visualization system relative to the target volume of ocular tissue during a movement of a focus of a laser beam to a depth location within the target volume of ocular tissue; and repeating the maintaining for each of a plurality of movements of the focus of the laser beam to different depth locations within the target volume of ocular tissue.

The present disclosure further relates to a system for imaging a region of an eye including a target volume of ocular tissue. The system includes a laser source configured to output a laser beam; a first optical subsystem configured to be coupled to the eye; a second optical subsystem optically coupled to the first optical subsystem, and a control system. The second optical subsystem includes: a) a focusing objective that is optically coupled to obtain the laser beam from the laser source, b) a visualization system that is optically coupled to the focusing objective, c) a first movement mechanism arranged to move the focusing objective and the visualization system, and d) a second movement mechanism arranged to move the visualization system. The control system is coupled to the second optical subsystem and configured to control the visualization system, the first movement mechanism, and the second movement mechanism to: move a visualization system to place a field of view of the visualization system at a location relative to the region of the eye; move the focusing objective to place a laser focus of a laser beam at a depth location within the target volume of ocular tissue; and move the visualization system without moving the focusing objective to keep the field of view of the visualization system at the location relative to the region of the eye.

The present disclosure further relates to a method of imaging a region of an eye including a target volume of ocular tissue. The method includes moving a visualization system to place a field of view of the visualization system at a location relative to the region of the eye; moving a focusing objective to place a laser focus of a laser beam at a depth location within the target volume of ocular tissue; and moving the visualization system without moving the focusing objective to keep the field of view of the visualization system at the location relative to the region of the eye.

It is understood that other aspects of apparatuses and methods will become apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### BRIEF DESCRIPTION OF THE DRAWINGS

Various aspects of systems, apparatuses, and methods will now be presented in the detailed description by way of example, and not by way of limitation, with reference to the accompanying drawings, wherein:

FIG. 1 is a sectional schematic illustration of a human eye and its interior anatomical structures.

FIG. 2 is a sectional schematic illustration of the irido-corneal angle of the eye of FIG. 1.

FIG. 3 is a sectional schematic illustration detailing anatomical structures in the irido-corneal angle of FIG. 2, including the trabecular meshwork, Schlemm's canal, and one or more collector channels branching from the Schlemm's canal.

FIG. 4 is a sectional schematic illustration of various outflow pathways for aqueous humor through the trabecular meshwork, Schlemm's canal, and collector channels of FIG. 3.

FIG. 5 is a sectional schematic illustration of a human eye showing various axes associated with the eye.

FIG. 6 is a sectional schematic illustration of an angled beam path along which one or more light beams may access the irido-corneal angle of the eye.

FIG. 7 is a block diagram of an integrated surgical system for non-invasive glaucoma surgery including a control system, a laser source, a visual observation apparatus, an optional OCT imaging apparatus, beam conditioners, scanners, and combiners, a focusing objective head, and a patient interface.

FIG. 8a is a detailed block diagram of an embodiment of the integrated surgical system of FIG. 7.

FIGS. 8b and 8c are detailed block diagrams of an embodiment of the integrated surgical system of FIG. 7 having a first optical subsystem that includes optical components of the focusing objective head and the patient interface, and a second optical subsystem that includes the visual observation apparatus, beam conditioners, scanners, and combiners, and a focusing objective of the focusing objective head.

FIGS. 9a and 9b are schematic illustrations of the focusing objective head of the integrated surgical system of FIG. 8b coupled to (FIG. 9a) and decoupled from (FIG. 9b) the patient interface of the integrated surgical system.

FIG. 9c is a schematic illustration of components of the focusing objective head and the patient interface included in FIGS. 9a and 9b.

FIGS. 10a and 10b are schematic illustrations of components of the integrated surgical system of FIGS. 7 and 8b functionally arranged to form a first optical system and a second optical subsystem that enable access to the irido-corneal angle along the angled beam path of FIG. 6.

FIG. 10c is a schematic illustration of a beam passing through the first optical subsystem of FIGS. 10a and 10b and into the eye.

FIG. 11 is a three-dimensional schematic illustration of anatomical structures in the irido-corneal angle, including the trabecular meshwork, Schlemm's canal, a collector channel branching from the Schlemm's canal, and a surgical volume of ocular tissue to be treated by the integrated surgical system of FIG. 7.

FIG. 12 is a two-dimensional schematic illustrations of anatomical structures in the irido-corneal angle and a three-dimensional laser treatment pattern to be applied by the integrated surgical system of FIG. 7 to affect a surgical volume of ocular tissue between the Schlemm's canal and the anterior chamber as shown in FIG. 11.

FIG. 13 is a three-dimensional schematic illustration of FIG. 11 subsequent to treatment of the surgical volume of ocular tissue by a laser based on the laser treatment pattern of FIG. 12 that forms an opening between the Schlemm's canal and the anterior chamber.

FIG. 14a is a schematic illustration of a three-dimensional laser treatment pattern formed by a number of stacked two-dimensional treatment planes or layers.

FIG. 14b is a schematic illustration of a two-dimensional treatment layer defined by an array of spots.

FIGS. 15a and 15b are schematic illustrations of two layers of a laser scanning process based on the treatment pattern of FIGS. 12, where the scanning begins at a shallow depth adjacent the anterior chamber and proceeds toward the Schlemm's canal.

FIGS. 16a and 16b are schematic illustrations of two layers of a laser scanning process based on the treatment pattern of FIGS. 12, where the scanning begins at a deep depth adjacent the Schlemm's canal and proceeds toward the anterior chamber.

FIG. 17 is a flowchart of a method imaging a region of an irido-corneal angle of an eye including a volume of ocular tissue targeted for laser treatment.

FIG. 18a is a schematic representation of a starting image obtained by the integrated surgical system of FIG. 8b while the focusing objective and visual observation apparatus are at a focus position.

FIGS. 18b and 18c are a schematic representations of subsequent images captured by the integrated surgical system of FIG. 8b after movement of the focusing objective and the visual observation apparatus in accordance with one or more aspects of this disclosure.

FIGS. 18d and 18e are a schematic representations of subsequent images captured by the integrated surgical system of FIG. 8b in the absence of movement of the focusing objective and the visual observation apparatus in accordance with one or more aspects of this disclosure.

FIG. 19 is a flowchart of the workflow of surgical steps enabled by the integrated surgical system of FIG. 8b to complete a laser cut in a region of interest.

FIG. 20 is a schematic block diagram of the control system of FIGS. 7 and 8b.

### DETAILED DESCRIPTION

Disclosed herein are systems and methods for imaging a region of an eye during laser treatment of a target volume of ocular tissue of the eye. The region of the eye may be, for example, the irido-corneal angle. The system includes a laser source configured to output a laser beam, a first optical subsystem configured to be coupled to the eye, a second optical subsystem optically coupled to the first optical subsystem, and a control system coupled to the second optical subsystem. The laser source may be a femtosecond laser source or a picosecond laser source. Such lasers provide non-thermal photo-disruption interaction with ocular tissue.

The second optical subsystem includes: a) a focusing objective that is optically coupled to obtain the laser beam from the laser source, b) a visualization system optically coupled to the focusing objective, c) a first movement mechanism arranged to move the focusing objective and the visualization system along a first axis, and d) a second movement mechanism arranged to move the visualization system along a second axis. In some embodiments, the first movement mechanism may include a first structure with which the focusing objective and the visualization system are associated, and a first motor configured to move the structure. In one configuration, the first structure is a plate, the focusing objective and the visualization system are mounted to the plate, and the first motor is a stepper motor that moves the plate (and thus the focusing objective and the visualization system) in incremental units associated with a scanning of the laser through the target volume of ocular tissue.

Similarly, the second movement mechanism may include a second structure with which the only visualization system is associated, and a second motor configured to move the second structure. In one configuration, the second structure is a plate, the visualization system is mounted to the plate, and the second motor is a stepper motor that moves the plate (and thus the visualization system) in incremental units associated with a scanning of the laser through the target volume of ocular tissue. Other mechanical arrangements for moving the focusing objective and the visualization system are contemplated. For example, the focusing objective may itself be associated with a motorized structure configured to move the objective only along the first axis, while the visualization system may itself be associated with a different motorized structure configured to move the visualization system along both the first axis and the second axis.

The control system is configured to control the visualization system, the first movement mechanism, and the second movement mechanism to: obtain a starting image of a region of an eye through the visualization system, place a laser focus of the laser beam at a depth location within the target volume of ocular tissue, and capture a subsequent image of the region of the eye through the visualization system while the laser focus is at the depth location. The visualization system is moved by the second movement mechanism such the subsequent image has a one or more image characteristics consistent with one or more corresponding characteristics of the starting image. These image characteristics may include, for example, field of view and one or more visual characteristics, such as quality of focus, magnification, resolution, contrast, and other quality factors (e.g., such as distortion and optical aberrations).

Femtosecond Laser Source

A surgical component of the integrated surgical system disclosed herein is a femtosecond laser. A femtosecond laser provides highly localized, non-thermal photo-disruptive laser-tissue interaction with minimal collateral damage to surrounding ocular tissue. Photo-disruptive interaction of the laser is utilized in optically transparent tissue. The principal mechanism of laser energy deposition into the ocular tissue is not by absorption but by a highly nonlinear multiphoton process. This process is effective only at the focus of the pulsed laser where the peak intensity is high. Regions where the beam is traversed but not at the focus are not affected by the laser. Therefore, the interaction region with the ocular tissue is highly localized both transversally and axially along the laser beam. The process can also be used in weakly absorbing or weakly scattering tissue. While femtosecond lasers with photo-disruptive interactions have been successfully used in ophthalmic surgical systems and commercialized in other ophthalmic laser procedures, none have been used in an integrated surgical system that accesses the irido-corneal angle.

In known refractive procedures, femtosecond lasers are used to create corneal flaps, pockets, tunnels, arcuate incisions, lenticule shaped incisions, partial or fully penetrating corneal incisions for keratoplasty. For cataract procedures the laser creates a circular cut on the capsular bag of the eye for capsulotomy and incisions of various patterns in the lens for breaking up the interior of the crystalline lens to smaller fragments to facilitate extraction. Entry incisions through the cornea opens the eye for access with manual surgical devices and for insertions of phacoemulsification devices and intra-ocular lens insertion devices. Several companies have commercialized such surgical systems, among them the IntraLase system now available from Johnson & Johnson Vision, Santa Ana, Calif., The LenSx and WaveLight systems from Alcon, Fort Worth, Tex., other surgical systems from Bausch and Lomb, Rochester, N.Y., Carl Zeiss Meditec AG, Germany, Ziemer, Port, Switzerland, and LENSAR, Orlando, Fla.

These existing systems are developed for their specific applications, for surgery in the cornea, and the crystalline lens and its capsular bag and are not capable of performing surgery in the irido-corneal angle 13 for several reasons. First, the irido-corneal angle 13 is not accessible with these surgical laser systems because the irido-corneal angle is too far out in the periphery and is outside of surgical range of these systems. Second, the angle of the laser beam from these systems, which is along the optical axis 24 to the eye 1, is not appropriate for reaching the irido-corneal angle 13, where there is significant scattering and optical distortion at the applied wavelength. Third, any imaging capabilities these systems may have do not have the accessibility, penetration depth and resolution to image the tissue along the trabecular outflow pathway 40 with sufficient detail and contrast.

In accordance with the integrated surgical system disclosed herein, clear access to the irido-corneal angle 13 is provided along the angled beam path 30. The tissue, e.g., cornea 3 and the aqueous humor 8 in the anterior chamber 7, along this angled beam path 30 is transparent for wavelengths from approximately 400 nm to 2500 nm and femtosecond lasers operating in this region can be used. Such mode locked lasers work at their fundamental wavelength with Titanium, Neodymium or Ytterbium active material. Non-linear frequency conversion techniques known in the art, frequency doubling, tripling, sum and difference frequency mixing techniques, optical parametric conversion can convert the fundamental wavelength of these lasers to practically any wavelength in the above-mentioned transparent wavelength range of the cornea.

Existing ophthalmic surgical systems apply lasers with pulse durations longer than 1 ns have higher photo-disruption threshold energy, require higher pulse energy and the dimension of the photo-disruptive interaction region is larger, resulting in loss of precision of the surgical treatment. When treating the irido-corneal angle 13, however, higher surgical precision is required. To this end, the integrated surgical system may be configured to apply lasers with pulse durations from 10 femtosecond (fs) to 1 nanosecond (ns) for generating photo-disruptive interaction of the laser beam with ocular tissue in the irido-corneal angle 13. While lasers with pulse durations shorter than 10 fs are available, such laser sources are more complex and more expensive. Lasers with the described desirable characteristics, e.g., pulse durations from 10 femtosecond (fs) to 1 nanosecond (ns), are commercially available from multiple vendors, such as Newport, Irvine, Calif., Coherent, Santa Clara, Calif., Amplitude Systems, Pessac, France, NKT Photonics, Birkerod, Denmark, and other vendors.

Visual Observation Apparatus

An imaging component of the integrated surgical system disclosed herein is a visual observation apparatus, also referred to herein as a visualization system. The visual observation apparatus may include, for example, a video camera, a telescope, and one or more illumination sources. The camera may be a digital camera fitted with a goniolens to provide gonioscopic images of the eye. The illumination sources are positioned for optimal irradiance of the object of interested, e.g., the irido-corneal angle of the eye including in particular, the trabecular meshwork. Illumination sources may be LEDs or light delivered via fiber optic cables. Illumination schemes are numerous: refractive ballistic schemes where the sources are placed in air and light refracts through the optics to reach the trabecular meshwork; transmissive ballistic schemes where illumination sources are inserted into pre-drilled holes or features inside lenses and adhered using index-matched epoxy; or reflective schemes where light from illumination sources strikes the trabecular meshwork after reflecting off designed reflective surfaces on lenses close to the eye.

OCT Imaging

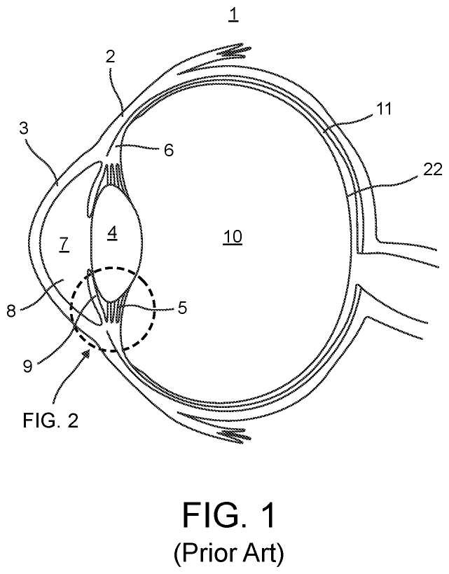

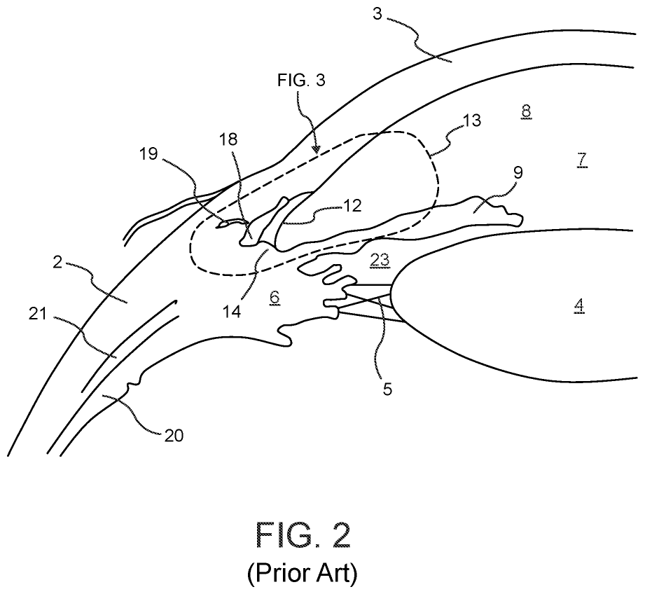

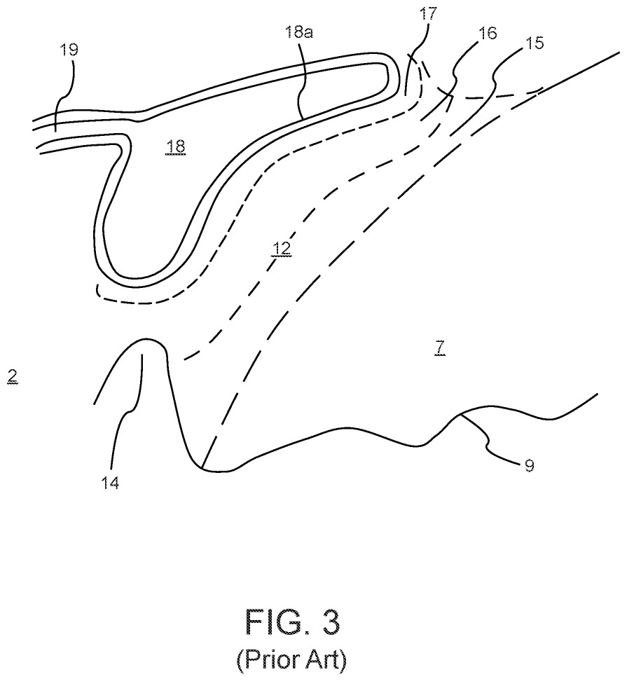

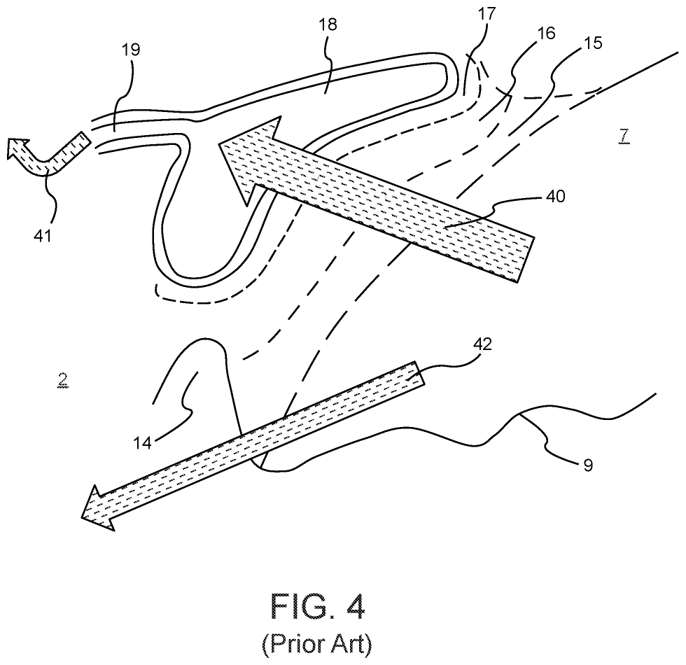

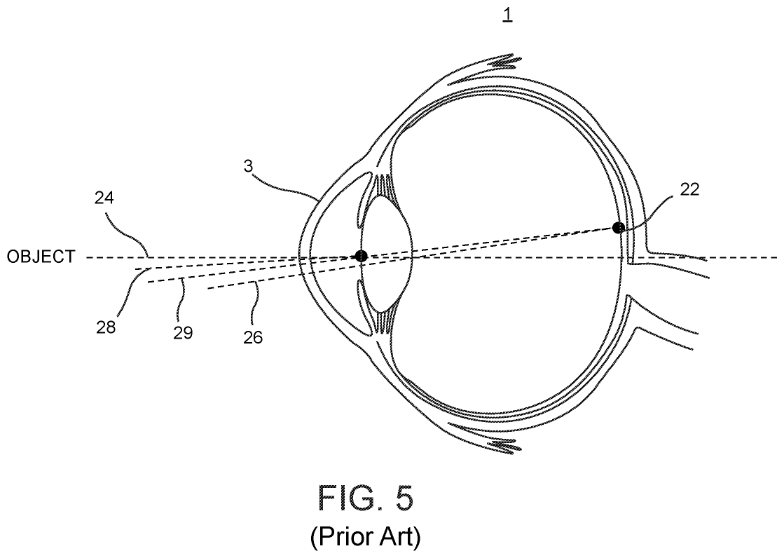

An optional imaging component of the integrated surgical system disclosed herein is an OCT imaging apparatus. OCT technology may be used to diagnose, locate, and guide laser surgery directed to the irido-corneal angle of the eye. For example, with reference to FIGS. 1-3, OCT imaging may be used to determine the structural and geometrical conditions of the anterior chamber 7, to assess possible obstruction of the trabecular outflow pathway 40 and to determine the accessibility of the ocular tissue for treatment. As previously described, the iris 9 in a collapsed anterior chamber 7 may obstruct and close off the flow of aqueous humor 8, resulting in closed-angle glaucoma. In open-angle glaucoma, where the macroscopic geometry of the angle is normal, the permeability of ocular tissue may be affected, by blockage of tissue along the trabecular outflow pathway 40 or by the collapse of the Schlemm's canal 18 or collector channels 19.

OCT imaging can provide the necessary spatial resolution, tissue penetration and contrast to resolve microscopic details of ocular tissue. When scanned, OCT imaging can provide two-dimensional (2D) cross-sectional images of the ocular tissue. As another aspect of the integrated surgical system, 2D cross-sectional images may be processed and analyzed to determine the size, shape, and location of structures in the eye for surgical targeting. It is also possible to reconstruct three-dimensional (3D) images from a multitude of 2D cross-sectional images but often it is not necessary. Acquiring, analyzing, and displaying 2D images is faster and can still provide all information necessary for precise surgical targeting.

OCT is an imaging modality capable of providing high resolution images of materials and tissue. Imaging is based on reconstructing spatial information of the sample from spectral information of scattered light from within the sample. Spectral information is extracted by using an interferometric method to compare the spectrum of light entering the sample with the spectrum of light scattered from the sample. Spectral information along the direction that light is propagating within the sample is then converted to spatial information along the same axis via the Fourier transform. Information lateral to the OCT beam propagation is usually collected by scanning the beam laterally and repeated axial probing during the scan. 2D and 3D images of the samples can be acquired this way. Image acquisition is faster when the interferometer is not mechanically scanned in a time domain OCT, but interference from a broad spectrum of light is recorded simultaneously. This implementation is called a spectral domain OCT. Faster image acquisition may also be obtained by scanning the wavelength of light rapidly from a wavelength scanning laser in an arrangement called a swept-source OCT.

The axial spatial resolution limit of the OCT is inversely proportional to the bandwidth of the probing light used. Both spectral domain and swept source OCTs are capable of axial spatial resolution below 5 micrometers (m) with sufficiently broad bandwidth of 100 nanometers (nm) or more. In the spectral domain OCT, the spectral interference pattern is recorded simultaneously on a multichannel detector, such as a charge coupled device (CCD) or complementary metal oxide semiconductor (CMOS) camera, while in the swept source OCT the interference pattern is recorded in sequential time steps with a fast optical detector and electronic digitizer. There is some acquisition speed advantage of the swept source OCT but both types of systems are evolving and improving rapidly, and resolution and speed is sufficient for purposes of the integrated surgical system disclosed herein. Stand-alone OCT systems and OEM components are now commercially available from multiple vendors, such as Optovue Inc., Fremont, Calif., Topcon Medical Systems, Oakland, N.J., Carl Zeiss Meditec AG, Germany, Nidek, Aichi, Japan, Thorlabs, Newton, N.J., Santec, Aichi, Japan, Axsun, Billercia, Mass., and other vendors.

Accessing the Irido-Corneal Angle

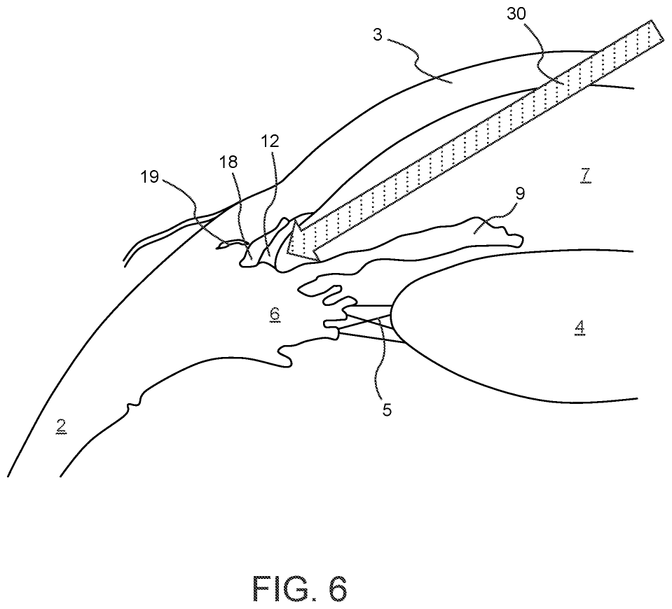

An important feature afforded by the integrated surgical system is access to the targeted ocular tissue in the irido-corneal angle 13. With reference to FIG. 6, the irido-corneal angle 13 of the eye may be accessed via the integrated surgical system along an angled beam path 30 passing through the cornea 3 and through the aqueous humor 8 in the anterior chamber 7. For example, one or more of an imaging beam, e.g., an OCT beam and/or a visual observation beam, and a laser beam may access the irido-corneal angle 13 of the eye along the angled beam path 30.

An optical system disclosed herein is configured to direct a light beam to an irido-corneal angle 13 of an eye along an angled beam path 30. The optical system includes a first optical subsystem and a second optical subsystem. The first optical subsystem includes a window formed of a material with a refractive index n_{w} and has opposed concave and convex surfaces. The first optical subsystem also includes an exit lens formed of a material having a refractive index nₓ. The exit lens also has opposed concave and convex surfaces. The concave surface of the exit lens is configured to couple to the convex surface of the window to define a first optical axis extending through the window and the exit lens. The concave surface of the window is configured to detachably couple to a cornea of the eye with a refractive index n_{c} such that, when coupled to the eye, the first optical axis is generally aligned with the direction of view of the eye.

The second optical subsystem is configured to output a light beam, e.g., an OCT beam or a laser beam. The optical system is configured so that the light beam is directed to be incident at the convex surface of the exit lens along a second optical axis at an angle α that is offset from the first optical axis. The respective geometries and respective refractive indices nₓ, and n_{w} of the exit lens and window are configured to compensate for refraction and distortion of the light beam by bending the light beam so that it is directed through the cornea 3 of the eye toward the irido-corneal angle 13. More specifically, the first optical system bends the light beam to that the light beam exits the first optical subsystem and enters the cornea 3 at an appropriate angle so that the light beam progresses through the cornea and the aqueous humor 8 in a direction along the angled beam path 30 toward the irido-corneal angle 13.

Accessing the irido-corneal angle 13 along the angled beam path 30 provides several advantages. An advantage of this angled beam path 30 to the irido-corneal angle 13 is that the OCT beam and laser beam passes through mostly clear tissue, e.g., the cornea 3 and the aqueous humor 8 in the anterior chamber 7. Thus, scattering of these beams by tissue is not significant. With respect to OCT imaging, this enables the use of shorter wavelength, less than approximately 1 micrometer, for the OCT to achieve higher spatial resolution. An additional advantage of the angled beam path 30 to the irido-corneal angle 13 through the cornea 3 and the anterior chamber 7 is the avoidance of direct laser beam or OCT beam light illuminating the retina 11. As a result, higher average power laser light and OCT light can be used for imaging and surgery, resulting in faster procedures and less tissue movement during the procedure.

Another important feature provided by the integrated surgical system is access to the targeted ocular tissue in the irido-corneal angle 13 in a way that reduces beam discontinuity. To this end, the window and exit lens components of the first optical subsystem are configured to reduce the discontinuity of the optical refractive index between the cornea 3 and the neighboring material and facilitate entering light through the cornea at a steep angle.

Having thus generally described the integrated surgical system and some of its features and advantages, a more detailed description of the system and its component parts follows.

Integrated Surgical System

In the following description, the term “beam” may—depending on the context—refer to one of a laser beam, an OCT beam, an illumination beam, an observation beam, an illumination/observation beam, or a visual beam. The term “colinear beams” refers to two or more different beams that are combined by optics of the integrated surgical system 1000 to share a same path to a same target location of the eye as they enter the eye. The term “non-colinear beams” refers to two or more different beams that have different paths into the eye. The term “co-targeted beams” refers to two or more different beams that have different paths into the eye but that target a same location of the eye. In colinear beams, the different beams may be combined to share a same path into the eye by dichroic or polarization beam splitters and delivered along a same optical path through a multiplexed delivery of the different beams. In non-colinear beams, the different beams are delivered into the eye along different optical paths that are separated spatially or by an angle between them. In the description to follow, any of the foregoing beams or combined beams may be generically referred to as a light beam. The terms distal and proximal may be used to designate the direction of travel of a beam, or the physical location of components relative to each other within the integrated surgical system. The distal direction refers to a direction toward the eye; thus, an OCT beam output by the OCT imaging apparatus moves in the distal direction toward the eye. The proximal direction refers to a direction away from the eye; thus, an OCT return beam from the eye moves in the proximal direction toward the OCT imaging apparatus.

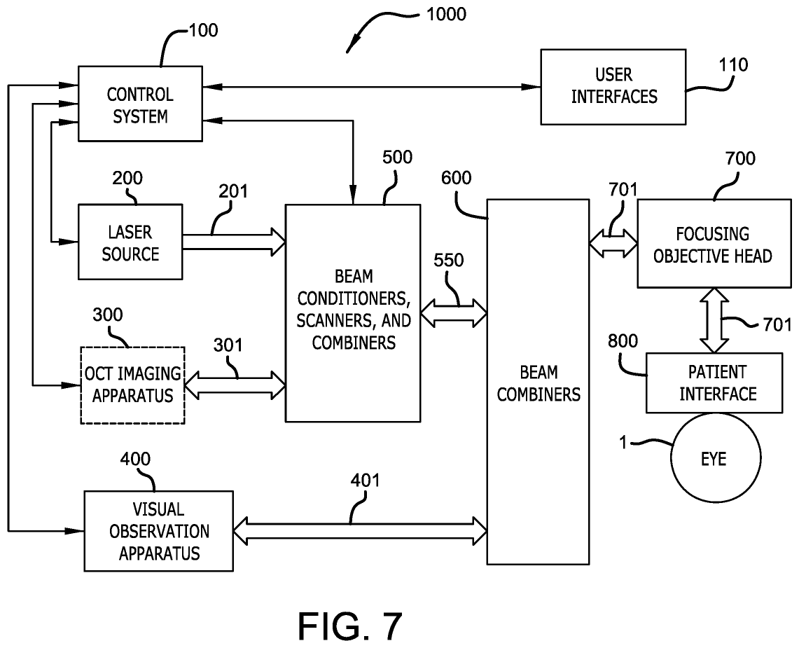

With reference to FIG. 7, an example integrated surgical system 1000 for non-invasive glaucoma surgery includes a control system 100, a surgical component 200, an optional first imaging component 300 and a second imaging component 400. In the embodiment of FIG. 7, the surgical component 200 is a femtosecond laser source, the optional first imaging component 300 is an OCT imaging apparatus, and the second imaging component 400 is a visual observation apparatus, e.g., a microscopic video camera, for direct viewing of anatomy of the eye. Other components of the integrated surgical system 1000 include beam conditioners, scanners, and combiners 500, beam combiners 600, a focusing objective head 700, and a patient interface 800.

The control system 100 may be a single computer or and plurality of interconnected computers configured to control the hardware and software components of the other components of the integrated surgical system 1000. A user interface 110 of the control system 100 accepts instructions from a user and displays information for observation by the user. Input information and commands from the user include but are not limited to system commands, motion controls for docking the patient's eye to the system, selection of pre-programmed or live generated surgical plans, navigating through menu choices, setting of surgical parameters, responses to system messages, determining and acceptance of surgical plans and commands to execute the surgical plan. Outputs from the system towards the user includes but are not limited to display of system parameters and messages, display of images of the eye, graphical, numerical, and textual display of the surgical plan and the progress of the surgery.

The control system 100 is connected to the other components 200, 300, 400, 500 of the integrated surgical system 1000. Control signals from the control system 100 to the femtosecond laser source 200 function to control internal and external operation parameters of the laser source, including for example, power, repetition rate and beam shutter. Control signals from the control system 100 to the optional OCT imaging apparatus 300 function to control OCT beam scanning parameters, and the acquiring, analyzing, and displaying of OCT images.

Laser beams 201 from the femtosecond laser source 200 and OCT beams 301 from the optional OCT imaging apparatus 300 are directed towards beam conditioners, scanners, and combiners 500. Beam conditioners set the basic beam parameters, beam size, divergence. Beam conditioners may also include additional functions, setting the beam power or pulse energy and shutter the beam to turn it on or off. Different kind of scanners can be used for the purpose of scanning the laser beam 201 and the OCT beam 301. For scanning transversal to a beam 201, 301, angular scanning galvanometer scanners are available for example from Cambridge Technology, Bedford, Mass., Scanlab, Munich, Germany.

To optimize scanning speed, the scanner mirrors are typically sized to the smallest size, which still support the required scanning angles and numerical apertures of the beams at the target locations. The ideal beam size at the scanners is typically different from the beam size of the laser beam 201 or the OCT beam 301, and different from what may be needed at an optical entrance of the focusing objective head 700. Therefore, beam conditioners may be applied before, after, or in between individual scanners. The beam conditioners, scanners, and combiners 500 includes scanners for scanning the beam transversally and axially. Axial scanning changes the depth of the focus at the target region. Axial scanning can be performed by moving a lens axially in the beam path with a servo or stepper motor.

Beam combiners, such as dichroic, polarization or other kind of beam combiners, collinearly combine the laser beam 201 and the OCT beam 301. In some embodiments, the laser beam 201 and the OCT beam 301 may be combined and then scanned using a common scanner. In other embodiments, the laser beam 201 and the OCT beam 301 beams may be scanned using separate scanners and then collinearly combined. In either case, a combined laser/OCT beam 550 is collinearly combined with an illumination beam 401 of the visual observation apparatus 400 with dichroic, polarization or other kind of beam combiners 600. The beam combiner 600 uses dichroic or polarization beam splitters to split and recombine light with different wavelength and/or polarization. The beam combiner 600 may also include optics to change certain parameters of the individual beams 201, 301, 401 such as beam size, beam angle and divergence. The combined laser/OCT/visual beam 701 is passed through optics of the focusing objective head 700 and optics of the patient interface 800 to reach a common target volume or surgical volume in the eye 1.

To resolve ocular tissue structures of the eye in sufficient detail, the imaging components 300, 400 of the integrated surgical system 1000 may provide an OCT beam 301 and a visual observation beam 401 having a spatial resolution of several micrometers. The resolution of the OCT beam 301 is the spatial dimension of the smallest feature that can be recognized in the OCT image. It is determined mostly by the wavelength and the spectral bandwidth of the OCT source, the quality of the optics delivering the OCT beam 301 to the target location in the eye, the numerical aperture of the OCT beam and the spatial resolution of the OCT imaging apparatus at the target location. In one embodiment, the OCT beam 301 of the integrated surgical system 1000 has a resolution of no more than 5 μm.

Likewise, the surgical laser beam 201 provided by the femtosecond laser source 200 may be delivered to targeted locations with several micrometer accuracy. The resolution of the laser beam 201 is the spatial dimension of the smallest feature at the target location that can be modified by the laser beam without significantly affecting surrounding ocular tissue. It is determined mostly by the wavelength of the laser beam 201, the quality of the optics delivering the laser beam to target location in the eye, the numerical aperture of the laser beam, the energy of the laser pulses in the laser beam and the spatial resolution of the laser scanning system at the target location. In addition, to minimize the threshold energy of the laser for photo-disruptive interaction, the size of the laser spot should be no more than approximately 5 μm.

It should be noted that, while the visual observation beam 401 is acquired by the visual observation apparatus 400 using fixed, non-scanning optics, the OCT beam 301 of the OCT imaging apparatus 300 is scanned laterally in two transversal directions. The laser beam 201 of the femtosecond laser source 200 is scanned in two lateral dimensions and the depth of the focus is scanned axially.

For practical embodiments, beam conditioning, scanning, and combining the optical paths are certain functions performed on the laser, OCT, and visual observation optical beams. Implementation of those functions may happen in a different order than what is indicated in FIG. 7. Specific optical hardware that manipulates the beams to implement those functions can have multiple arrangements with regards to how the optical hardware is arranged. They can be arranged in a way that they manipulate individual optical beams separately, in another embodiment one component may combine functions and manipulates different beams. For example, a single set of scanners can scan both the laser beam 201 and the OCT beam 301. In this case, separate beam conditioners set the beam parameters for the laser beam 201 and the OCT beam 301, then a beam combiner combines the two beams for a single set of scanners to scan the beams. While many combinations of optical hardware arrangements are possible for the integrated surgical system, the following section describes in detail an example arrangement.

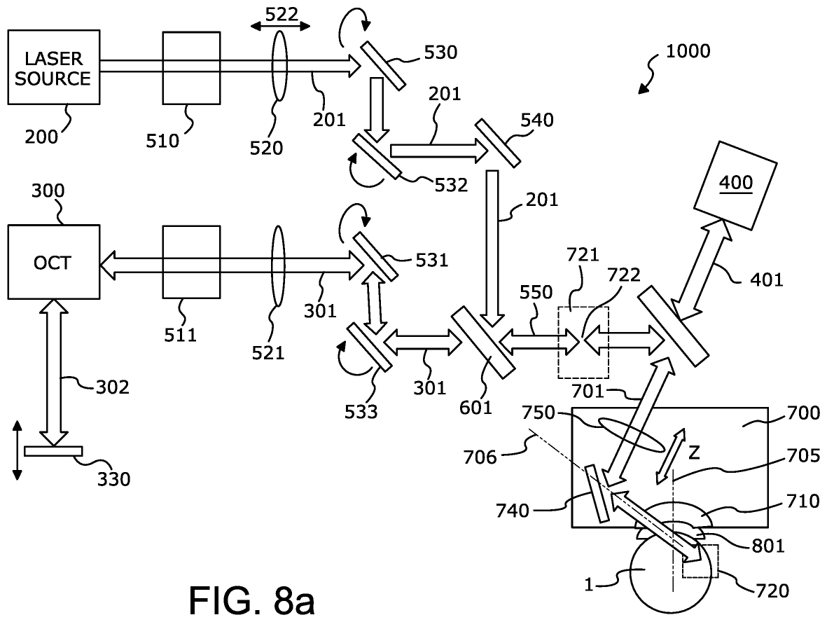

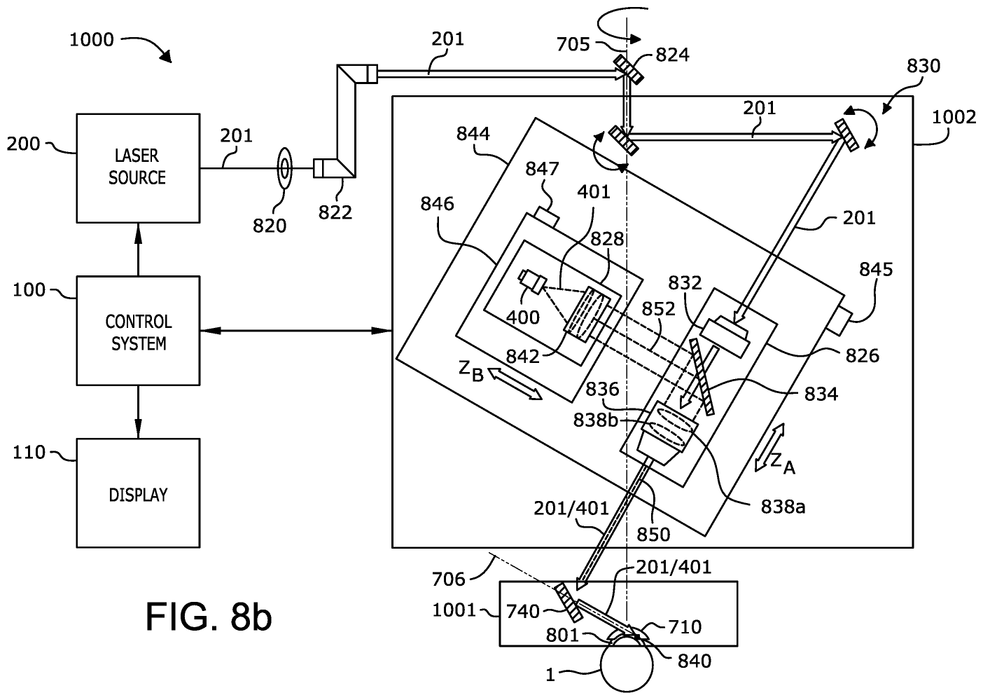

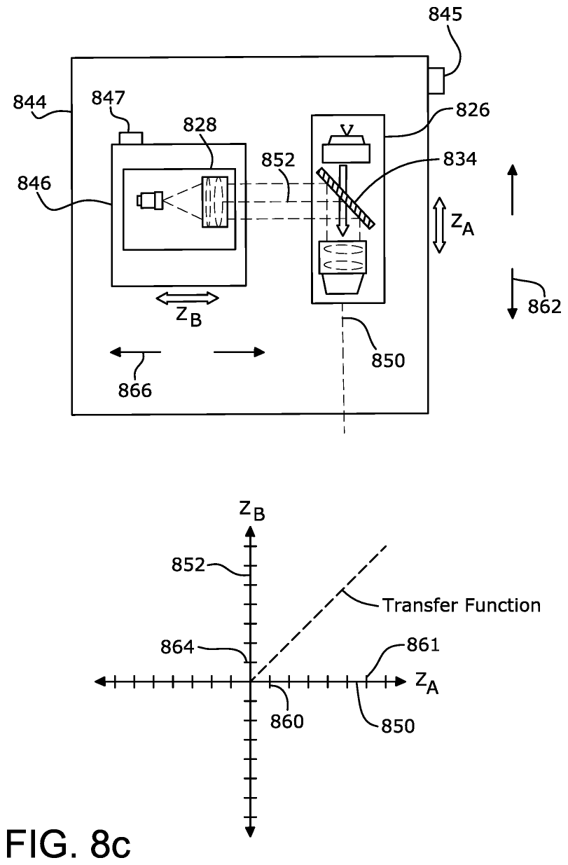

Referring to FIG. 8a, in some embodiments an integrated surgical system 1000 includes optical subsystems configured together to deliver each of a laser beam 201, an OCT beam 301, and an illumination beam 401 in the distal direction toward an eye 1, and receive each of an OCT return beam and an observation beam 401 back from the eye 1.

Regarding the delivery of a laser beam, a laser beam 201 output by the femtosecond laser source 200 passes through a beam conditioner 510 where the basic beam parameters, beam size, divergence are set. The beam conditioner 510 may also include additional functions, setting the beam power or pulse energy and shutter the beam to turn it on or off. After existing the beam conditioner 510, the laser beam 210 enters an axial scanning lens 520. The axial scanning lens 520, which may include a single lens or a group of lenses, is movable in the axial direction 522 by a servo motor, stepper motor or other control mechanism. Movement of the axial scanning lens 520 in the axial direction 522 changes the axial distance of the focus of the laser beam 210 at a focal point.

In accordance with a particular embodiment of the integrated surgical system 1000, an intermediate focal point 722 is set to fall within, and is scannable in, the conjugate surgical volume 721, which is an image conjugate of the surgical volume 720, determined by optics of the focusing objective head 700. The surgical volume 720 is the spatial extent of the region of interest within the eye where imaging and surgery is performed. For glaucoma surgery, the surgical volume 720 is the vicinity of the irido-corneal angle 13 of the eye 1.

A pair of transverse scanning mirrors 530, 532 rotated by a galvanometer scanner scan the laser beam 201 in two essentially orthogonal transversal directions, e.g., in the x and y directions. Then the laser beam 201 is directed towards a dichroic or polarization beam splitter 540 where it is reflected toward a beam combining mirror 601 configured to combine the laser beam 201 with an OCT beam 301.

Regarding delivery of an OCT beam, an OCT beam 301 output by the OCT imaging apparatus 300 passes through a beam conditioner 511, an axially moveable focusing lens 521 and a transversal scanner with scanning mirrors 531 and 533. The focusing lens 521 is used set the focal position of the OCT beam in the conjugate surgical volume 721 and the real surgical volume 720. The focusing lens 521 is not scanned for obtaining an OCT axial scan. Axial spatial information of the OCT image is obtained by Fourier transforming the spectrum of the interferometrically recombined OCT return beam 301 and reference beams 302. However, the focusing lens 521 can be used to re-adjust the focus when the surgical volume 720 is divided into several axial segments. This way the optimal imaging spatial resolution of the OCT image can be extended beyond the Rayleigh range of the OCT beam 301, at the expense of time spent on scanning at multiple ranges.

Proceeding in the distal direction toward the eye 1, after the scanning mirrors 531 and 533, the OCT beam 301 is combined with the laser beam 201 by the beam combiner mirror 601. The OCT beam 301 and laser beam 201 components of the combined laser/OCT beam 550 are multiplexed and travel in the same direction to be focused at an intermediate focal point 722 within the conjugate surgical volume 721. After having been focused in the conjugate surgical volume 721, the combined laser/OCT beam 550 propagates to a second beam combining mirror 602 where it is combined with a visual observation beam 401 to form a combined laser/OCT/visual beam 701.

The combined laser/OCT/visual beam 701 traveling in the distal direction then passes through a relay lens 750 included in the focusing objective head 700, is reflected by a reflecting surface 740, which may be a planar beam-folding mirror or a facet inside an optic, and then passes through an exit lens 710 of the focusing objective head and a window 801 of a patient interface, where the intermediate focal point 722 of the laser beam within the conjugate surgical volume 721 is re-imaged into a focal point in the surgical volume 720. The optics of the focusing objective head 700 re-images the intermediate focal point 722, through the window 801 of the patient interface, into the ocular tissue within the surgical volume 720. In one configuration, the reflecting surface 740 in the form of a facet inside an optic may have a specialized coating for broadband reflection (visible, OCT and femtosecond) and low difference between s and p polarization group delay dispersion (GDD).

A scattered OCT return beam 301 from the ocular tissue travels in the proximal direction to return to the OCT imaging apparatus 300 along the same paths just described, in reverse order. The reference beam 302 of the OCT imaging apparatus 300, passes through a reference delay optical path and return to the OCT imaging apparatus from a moveable mirror 330. The reference beam 302 is combined interferometrically with the OCT return beam 301 on its return within the OCT imaging apparatus 300. The amount of delay in the reference delay optical path is adjustable by moving the moveable mirror 330 to equalize the optical paths of the OCT return beam 301 and the reference beam 302. For best axial OCT resolution, the OCT return beam 301 and the reference beam 302 are also dispersion compensated to equalize the group velocity dispersion within the two arms of the OCT interferometer.

When the combined laser/OCT/visual beam 701 is delivered through the cornea 3 and the anterior chamber 7, the combined beam passes through posterior and anterior surface of the cornea at a steep angle, far from normal incidence. These surfaces in the path of the combined laser/OCT/visual beam 701 may create excessive astigmatism and coma aberrations that need to be compensated for.

Referring to FIG. 8b, in some embodiments disclosed herein an integrated surgical system 1000 for treating a target volume of ocular tissue includes a control system 100, a laser source 200, a first optical subsystem 1001, and a second optical subsystem 1002. These components are arranged and configured together to deliver a laser beam 201 and an illumination beam 401 in the distal direction toward a target volume of ocular tissue of an eye 1, and receive an observation beam 401 back from the eye 1. The delivered illumination beam and received observation beam may collectively be referred to herein as an illumination/observation beam 401.

The first optical subsystem 1001 includes optics configured to establish a common optical axis 706 or common optical path through the cornea and the anterior chamber into the irido-corneal angle for a laser beam 201 and an illumination/observation beam 401. The common optical axis 706 is offset from a first optical axis 705 within the direction of view. In one configuration, the first optical subsystem 1001 includes a reflecting surface 740 in the form of a beam-folding mirror, an exit lens 710, and a window 801. In another configuration, the first optical subsystem 1001 includes a facet inside an optic that has a specialized coating for broadband reflection (visible and femtosecond) and low difference between s and p polarization group delay dispersion (GDD), an exit lens 710 associated with the optic, and a window 801.

The second optical subsystem 1002 includes a scanner component 830, a focusing objective 826, and a visualization system 828. The scanner component 830 is arranged and configured to receive a laser beam 201 from the laser source 200 and to optically couple the laser beam with the focusing objective 826. The scanner component 830 may include a pair of galvanometer scanning mirrors. In some embodiments the second optical subsystem 1002 is configured to rotate relative to the first optical axis 705. This rotation enables optical access to the whole 360-degree circumference of the irido-corneal angle 13 of the eye 1.

The focusing objective 826 is arranged and configured to receive the laser beam 201 from the scanner component 830 and to optically couple the laser beam with the first optical subsystem 1001. To this end, the focusing objective 826 aligns the laser beam 201 with the reflecting surface 740, which in turn aligns the laser beam along the common optical axis 706. In some embodiments the focusing objective 826 includes focusing optics, e.g., a back-end objective 832 having two lenses (not shown) and a front-end objective 836 with a first objective lens 838a and a second objective lens 838b, and a dichroic mirror 834 located between the back-end objective 832 and the front-end objective 836.

The focusing objective 826 is mounted on a first structure, e.g., a first plate 844, that is instrumented with a first motor, e.g., an objective motor 845, configured to move the focusing objective back and forth along a first mechanical axis 850 in the Z_{A} direction, to thereby adjust the position of the focus of the laser beam 201 along the common optical axis 706. The first mechanical axis 850 may be aligned with, e.g., co-axial with or parallel to the central, optical axis of the focusing objective 826.

The visualization system 828, which in some embodiments includes a visual observation apparatus 400 and a telescope 842, is arranged and configured to optically couple an illumination beam 401 with the focusing objective 826. To this end, the visual observation apparatus 400 of the visualization system 828 may include an illumination source that provides the illumination beam 401. In other configurations (not shown in FIG. 8b), the illumination source may be a component of the visualization system 828 that is separate from the visual observation apparatus 400. In either case, the optical output of the visualization system 828 is aligned with the dichroic mirror 834 of the focusing objective 826, and thereby couples the illumination beam 401 with the dichroic mirror. The dichroic mirror 834 may collinearly combine the laser beam 201 and the illumination beam 401. The dichroic mirror 834 aligns the collinearly combined laser/illumination beam 201/401 with the reflecting surface 740 of the first optical subsystem 1001, which in turn aligns the laser/illumination beam along the common optical axis 706.

The visualization system 828 is mounted on a second structure, e.g., a second plate 846, that is instrumented with a second motor, e.g., a camera motor 847, configured to move the visualization system back and forth along a second mechanical axis 852 in the Z_{B} direction, to thereby adjust the position of the depth of field of the visualization system 828 along the common optical axis 706. The second mechanical axis 852 may be aligned with, e.g., co-axial with or parallel to the central, optical axis of the visualization system 828. The second plate 846 is also mounted on the first plate 844. Accordingly, the visualization system 828 moves together with the focusing objective 826 in the Z_{A} direction upon movement of the first plate 844. This movement maintains the alignment of the visualization system 828 with the dichroic mirror 834 of the focusing objective 826, and thereby maintains optical coupling between the visualization system and the focusing objective.

Additionally, through its association with the second plate 846, the visualization system 828 may also move in the Z_{B} direction without any corresponding movement by the focusing objective 826. As will be described further below, the combination of: 1) movement of the visualization system 828 and movement of the focusing objective 826 along the first mechanical axis 850 in the Z_{A} direction, and 2) movement of the visualization system by itself along the second mechanical axis 852 in the Z_{B} direction without corresponding movement of the focusing objective, provides for fixed, stable, real-time imaging of the target volume of ocular tissue during laser scanning, wherein the field of view and depth of field of the image are consistent.

In the integrated surgical system 1000 of FIG. 8b, the focusing objective 826 and the visualization system 828 are arranged such that the first mechanical axis 850 (or corresponding optical axis) of the focusing objective is orthogonal with the second mechanical axis 852 (or corresponding optical axis) of the visualization system. This arrangement, also referred to herein as the “geometry,” of the optical axes of the focusing objective 826 and the visualization system 828 is merely one example geometry. Numerous other geometries are possible. For example, the focusing objective 826 and the visualization system 828 may be arranged such that the first mechanical axis 850 and the second mechanical axis 852 are at any angle relative to each other, are parallel to each other, or are co-axial (i.e., they share the same mechanical axis).

Continuing with reference to FIG. 8b, the laser source 200 is configured to emit a laser beam 201 of femtosecond light pulses through a control shutter 820 and into an articulated arm 822. The articulated arm 822 transmits the laser beam 201 onto a turning mirror 824. The fixed turning mirror 824 is separate from the second optical subsystem 1002 and is arranged relative to the laser source 200 and the second optical subsystem to direct a laser beam 201 into the scanner component 830 of the second optical subsystem. The scanner component 830, in turn directs the laser beam 201 into the back-end objective 832 of the focusing objective 826.

The back-end objective 832 functions as an expanding telescope that increases the diameter of the laser beam 201 that, together with other components and aspects of the optical design, pre-compensates for aberrations, e.g., astigmatism, coma, spherical aberration, which will be introduced by the human cornea. The expanded laser beam 201 is then incident onto the dichroic mirror 834. The dichroic mirror 834 is configured to transmit the laser wavelength, e.g., 1030 nm in the case of a femtosecond laser, and to not reflect the laser wavelength. The expanded laser beam is then incident into the first objective lens 838a included in the front-end objective 836 of the focusing objective 826. The laser beam 201 continues to diverge as it passes through the first objective lens 838a. The laser beam 201 is then incident into the second objective lens 838b. The laser beam 201 exits the second objective lens 838b and enters the first optical subsystem 1001.

Once in the first optical subsystem 1001, the laser beam 201 is directed into a reflective surface 740 in the form of a beam-folding mirror, through the exit lens 710 and the window 801, and into the irido-corneal angle. In the case of a reflective surface 740 in the form of a facet in an optic that has a specialized coating for broadband reflection (visible and femtosecond) and low difference between s and p polarization GDD, the laser beam 201 refracts through a convex surface of the optic, strikes the facet and internally reflects through the optic and then exits through a concave surface corresponding to the exit lens 710. The laser beam 201 then passes through the window 801, the cornea, and the anterior chamber, and to a spot 840 in the irido-corneal angle at a predetermined distance from the exit surface of the second objective lens 838b.

Regarding the diameter of the expanded laser beam 201, the diameter of the beam when it enters the first objective lens 838a in the front-end objective 836 determines the final numerical aperture of the laser beam. The larger the diameter of the expanded laser beam 201, the greater the numerical aperture. In general, laser beams 201 with greater numerical apertures are focused onto smaller spots 840. The beam diameter and the numerical aperture are design choices that depend on the intended use of the device. In the integrated surgical system 1000, the diameter of the expanded laser beam is about 6 mm.

Continuing with reference to FIG. 8b, the visualization system 828 enables visualization of anatomy of the eye 1, including the circumferential angle. As previously mentioned, the visualization system 828 may include a visual observation apparatus 400 and a telescope 842. In one configuration, the visual observation apparatus 400 includes an illumination source and a gonioscopic video camera. The illumination source component of the visual observation apparatus 400 shines an illumination beam 401 through the telescope 842 and onto the dichroic mirror 834. The illumination beam 401 reflects from the dichroic mirror 834 into the first objective lens 838a included in the front-end objective 836. The illumination beam 401 passes through the first objective lens 838a and into the second objective lens 838b. The second objective lens 838b focuses visible light from the illumination beam 401 to illuminate an area within the circumferential angle. The illumination beam 401 exits the second objective lens 838b and enters the first optical subsystem 1001.

Once in the first optical subsystem 1001, the illumination beam 401 may be directed into the reflective surface 740, through the exit lens 710 and the window 801, and into the irido-corneal angle. The illumination beam 401 diffuses or scatters inside the target volume of ocular tissue and back-scattered light exits the tissue as a returning observation beam. The returning observation beam 401 reflects off the reflective surface 740 and returns to the second optical subsystem 1002 through the front-end objective 836 and onto the second dichroic mirror 834. The returning observation beam 401 of light enters the visualization system 828 where it is focused by the telescope 842 onto the video camera component of the visual observation apparatus 400 where the observation beam 401 impinges a sensor to form an image of the circumferential angle.

In accordance with embodiments disclosed herein, the visualization system 828 is positioned during laser scanning such that the depth of field of the visualization system is fixed relative to an image object, e.g., the surface of the trabecular meshwork where a target volume of ocular tissue is located. As such, the image resulting from impingement of the observation beam 401 on the sensor of the visualization system 828 is fixed. In other words, the mapping between the points of the image object and the points on the sensor of the visualization system 828 are fixed during scanning. Accordingly, the field of view of the captured image is fixed and image quality is consistent.

As disclosed further below, the visualization system 828 has a depth of field greater than the thickness of the target volume of ocular tissue. For example, in some embodiments, the depth of field is in the range of 1.5 mm to 3.0 mm in tissue. This design feature of the visualization system 828 maintains image quality during scanning. Other optical specifications of the visualization system 828 include: 1) a resolution capable of resolving features of a certain size, e.g., less than 10 μm, 2) illumination uniformity, meaning illumination coverage is uniform and does not have hot spots or uneven light intensity, and 3) a field of view (FOV) greater that 60 degrees so that a surgeon can see enough of the relevant anatomy.

Although not shown in FIG. 8b, the second optical subsystem 1002 may include an OCT imaging apparatus similar to that describe with reference to FIG. 8a. In such a configuration, an OCT beam output by an OCT imaging apparatus may be optically, collinearly combined with the laser beam 201 by additional optics (not shown) of the second optical subsystem 1002. An example of a second optical subsystem configured as such is disclosed in U.S. Patent Application Publication No. US 2020/0352785, the entire disclosure of which is hereby incorporated by reference.

The second optical subsystem 1002 may also include a dual beam apparatus for use in detecting a surface of ocular tissue in the target volume of ocular tissue. In such a configuration, dual aiming beams output by a dual beam apparatus may be optically, collinearly combined with the laser beam 201 by additional optics (not shown) of the second optical subsystem 1002. An example of a second optical subsystem configured as such is also disclosed in U.S. Patent Application Publication No. US 2020/0352785.

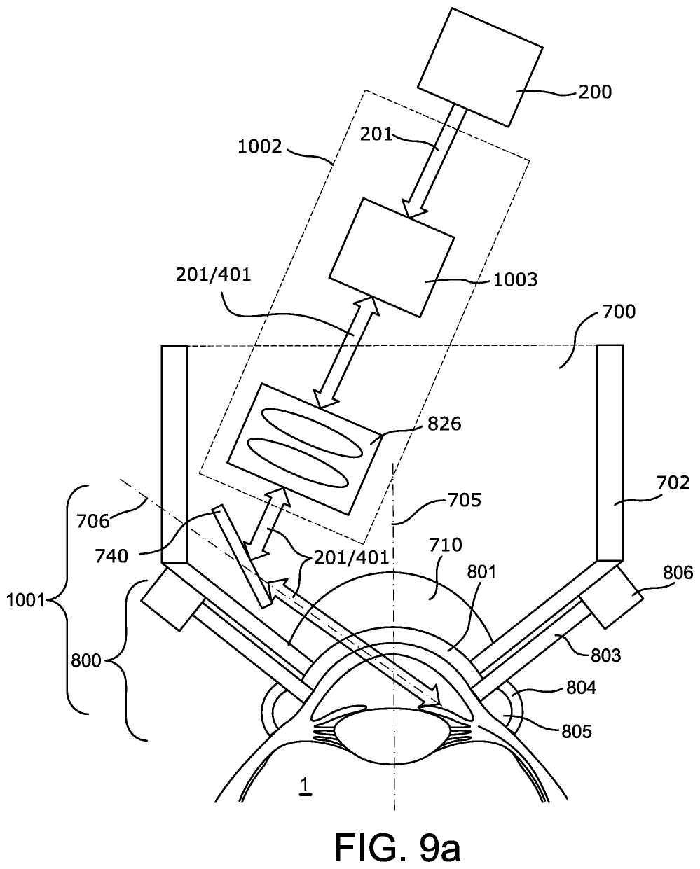

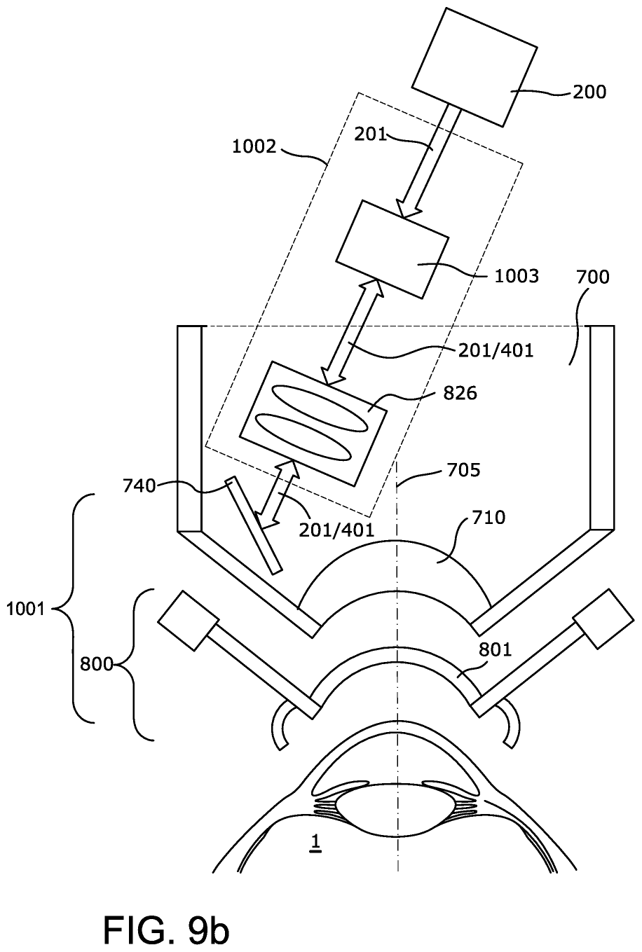

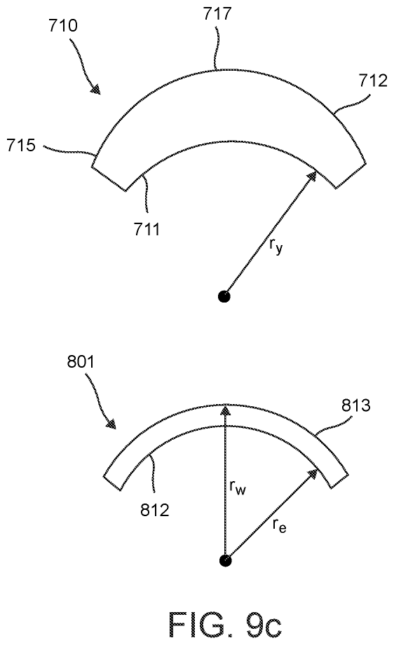

With reference to FIGS. 9a and 9b, considering further the embodiment of the integrated surgical system 1000 described with reference to FIG. 8b, components of the first optical subsystem 1001 and the second optical subsystem 1002 may be included in one or more of the focusing objective head 700 and the patient interface 800 of the integrated surgical system. For example, as shown in FIGS. 9a and 9b, the window 801 of the first optical subsystem 1001 may be included in the patient interface 800, while the exit lens 710 and the reflecting surface 740 of the first optical subsystem and the focusing objective 826 of the second optical subsystem 1002 may be included in the focusing objective head 700. FIG. 9a shows the eye 1, the patient interface 800 and the focusing objective head 700 coupled together. FIG. 9b shows the eye 1, the patient interface 800 and the focusing objective head 700 detached from one another.

The patient interface 800 optically and physically couples the eye 1 to the focusing objective head 700, which in turn optically couples with other optic components of the integrated surgical system 1000. The patient interface 800 serves multiple functions. It immobilizes the eye relative to components of the integrated surgical system; creates a sterile barrier between the components and the patient; and provides optical access between the eye and the instrument. The patient interface 800 is a sterile, single use disposable device and it is coupled detachably to the eye 1 and to the focusing objective head 700 of the integrated surgical system 1000.

The patient interface 800 includes the window 801 of the first optical subsystem 1001. The window 801 has an eye-facing, concave surface 812 and an objective-facing, convex surface 813 opposite the concave surface. The window 801 thus has a meniscus form. With reference to FIG. 9c, the concave surface 812 is characterized by a radius of curvature re, while the convex surface 813 is characterized by a radius of curvature r_{w}. The concave surface 812 is configured to couple to the eye, either through a direct contact or through index matching material, liquid, or gel, placed in between the concave surface 812 and the eye 1.

The window 801 may be formed of glass and has a refractive index n_{w}. In one embodiment, the window 801 is formed of fused silica and has a refractive index n_{w} of 1.45. Fused silica has the lowest index from common inexpensive glasses. Fluoropolymers such as the Teflon AF are another class of low index materials that have refractive indices lower than fused silica, but their optical quality is inferior to glasses, and they are relatively expensive for high volume production. In another embodiment the window 801 is formed of the common glass BK7 and has a refractive index n_{w} of 1.50. A radiation resistant version of this glass, BK7G18 from Schott AG, Mainz, Germany, allows gamma sterilization of the patient interface 800 without the gamma radiation altering the optical properties of the window 801.

Returning to FIGS. 9a and 9b, the window 801 is surrounded by a wall 803 of the patient interface 800 and an immobilization device, such as a suction ring 804. When the suction ring 804 is in contact with the eye 1, an annular cavity 805 is formed between the suction ring and the eye. When vacuum applied to the suction ring 804 and the cavity via a vacuum tube a vacuum pump (not shown in FIGS. 9a and 9b), vacuum forces between the eye and the suction ring attach the eye to the patient interface 800 during surgery. Removing the vacuum releases or detach the eye 1.

The end of the patient interface 800 opposite the eye 1 includes an attachment interface 806 configured to attach to the housing 702 of the focusing objective head 700 to thereby affix the position of the eye relative to the other components of the integrated surgical system 1000. The attachment interface 806 can work with mechanical, vacuum, magnetic or other principles and it is also detachable from the integrated surgical system.

The focusing objective head 700 includes the exit lens 710 of the first optical subsystem 1001. The exit lens 710 may also be referred to herein as the superdome. In one configuration, the exit lens 710 is an aspheric lens having an eye-facing, concave surface 711 and a convex surface 712 opposite the concave surface. The exit lens 710 thus has a meniscus form. While the exit lens 710 shown in FIGS. 9a and 9b is an aspheric lens giving more design freedom, in other configurations the exit lens may be a spherical lens. Alternatively, constructing the exit lens 710 as a compound lens, as opposed to a singlet, allows more design freedom to optimize the optics while preserving the main characteristics of the optical system as presented here.

With reference to FIG. 9c, the concave surface 711 of the exit lens 710 is characterized by a radius of curvature r_{y}, while the convex surface 712 is characterized by an aspheric shape. The aspheric convex surface 712 in combination with the spherical concave surface 711 result in an exit lens 710 having varying thickness, with the outer perimeter edges 715 of the lens being thinner than the central, apex region 717 of the lens. The concave surface 711 is configured to couple to the convex surface 813 of the window 801. In one embodiment, the exit lens 710 is formed of fused silica and has a refractive index nₓ of 1.45.

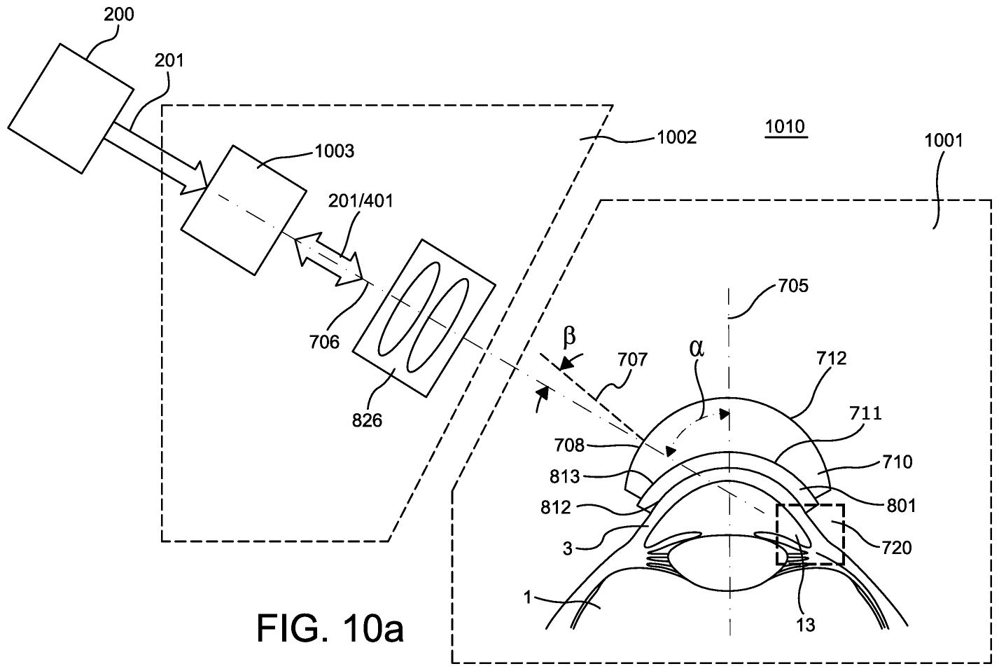

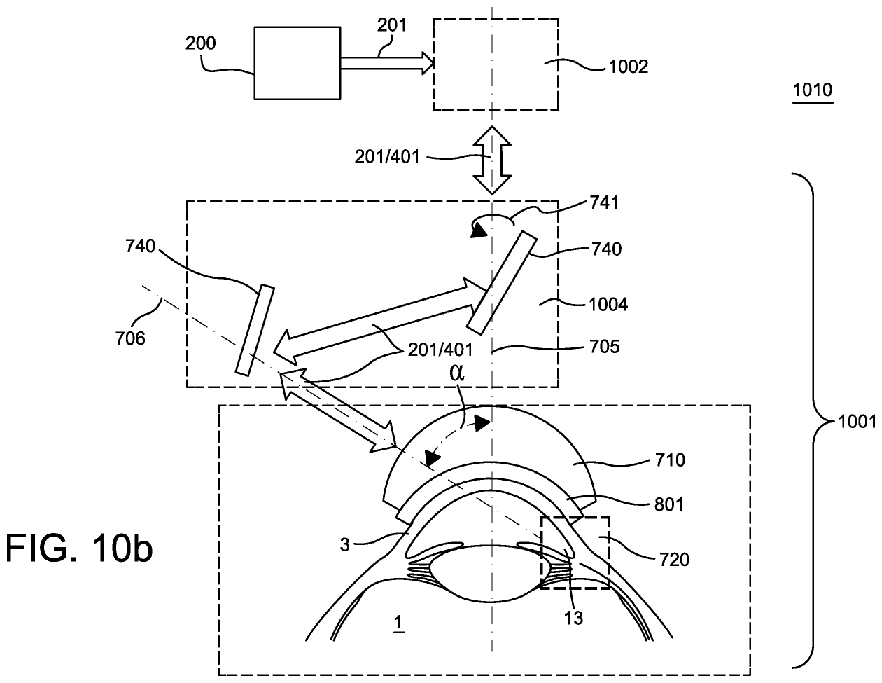

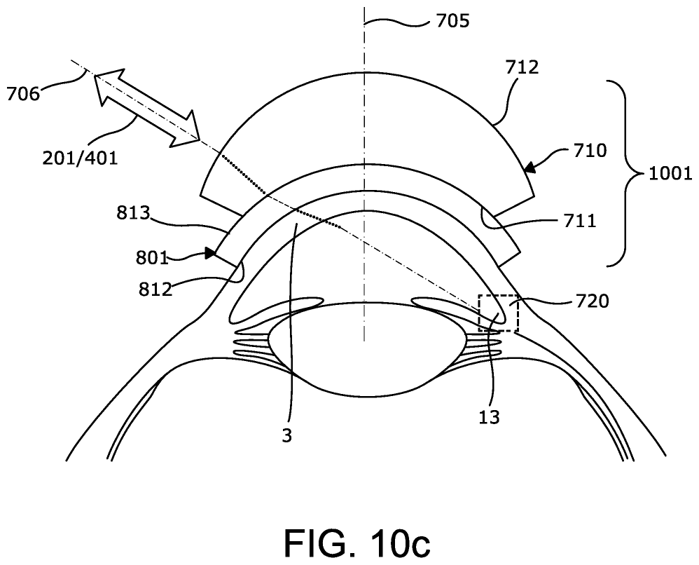

FIGS. 10a and 10b are schematic illustrations of different configurations of an optical system 1010 of the integrated surgical system 1000 of FIG. 8b. Each configuration of an optical system 1010 includes the first optical subsystem 1001 and the second optical subsystem 1002 of FIG. 8b. FIG. 10c is a schematic illustration of a laser/visual beam 201/401 passing through the first optical subsystem 1001 of FIGS. 10a and 10b. For simplicity of illustration, all components of the first optical subsystem 1001 and the second optical subsystem 1002 are not included in FIGS. 10a and 10b. Also, for additional simplicity in FIG. 10a, the reflecting surface 740 of the first optical subsystem 1001 is not included and the combined laser/visual beam 201/401 shown in FIG. 9a is unfolded or straightened out. It is understood by those skilled in the art that adding or removing planar beam folding mirrors does not alter the principal working of the optical system 1010 formed by the first optical subsystem 1001 and the second optical subsystem 1002.

With reference to FIG. 10a, the first optical subsystem 1001 of the integrated surgical system 1000 includes the exit lens 710 of a focusing objective head 700 and the window 801 of a patient interface 800. The exit lens 710 and the window 801 are arranged relative to each other to define a first optical axis 705. The first optical subsystem 1001 is configured to receive a beam, e.g., a combined laser/visual beam 201/401, incident at the convex surface 712 of the exit lens 710 along a second optical axis 706, and to direct the beam toward a surgical volume 720 in the irido-corneal angle 13 of the eye.

During a surgical procedure, all, or a portion of the first optical subsystem 1001 may be assembled by interfacing the convex surface 813 of the window 801 with the concave surface 711 of the exit lens 710. To this end, a focusing objective head 700 is docked together with a patient interface 800. As a result, the concave surface 711 of the exit lens 710 is coupled to the convex surface 813 of the window 801. The coupling may be by direct contact or through a layer of index matching fluid. For example, when docking the patient interface 800 to the focusing objective head 700, a drop of index matching fluid can be applied between the contacting surfaces to eliminate any air gap that may be between the two surfaces 711, 813 to thereby help pass the laser/visual beam 201/401 through the gap with minimal Fresnel reflection and distortion.

In order to direct the beam toward a surgical volume 720 of ocular tissue in the irido-corneal angle 13 of the eye, the first optical subsystem 1001 is designed to account for refraction of the beam 201/401 as it passes through the exit lens 710, the window 801 and the cornea 3. To this end, and with reference to FIG. 10c, the refractive index nₓ of the exit lens 710 and the refractive index n_{w} of the window 801 are selected in view of the refractive index n_{c} of the cornea 3 to cause appropriate beam bending through the first optical subsystem 1001 so that when the laser/visual beam 201/401 exits the subsystem and passes through the cornea 3, the beam path is generally aligned to fall within the irido-corneal angle 13.

Continuing with reference to FIG. 10c and beginning with the interface between the window 801 and the cornea 3. Too steep of an angle of incidence at the interface where the laser/visual beam 201/401 exits the window 801 and enters the cornea 3, i.e., at the interface between the concave surface 812 of the window and the convex surface of the cornea 3, can create excessive refraction and distortion. To minimize refraction and distortion at this interface, in one embodiment of the first optical subsystem 1001, the refractive index of the window 801 is closely matched to the index of the cornea 3. For example, as describe above with reference to FIGS. 9a and 9b, the window 801 may have a refractive index lower than 1.42 to closely match the cornea 3, which has a refractive index of 1.36.

Excessive refraction and distortion at the interface where the combined laser/visual beam 201/401 exits the window 801 and enters the cornea 3 may be further compensated for by controlling the bending of the beam as it passed through the exit lens 710 and the window 801. To this end, in one embodiment of the first optical subsystem 1001 the index of refraction n_{w} of the window 801 is larger than each of the index of refraction nₓ of the exit lens 710 and the index of refraction n_{c} of the cornea 3. As a result, at the interface where the combined laser/visual beam 201/401 exits the exit lens 710 and enters the window 801, i.e., interface between the concave surface 711 of the exit lens and the convex surface 813 of the window, the beam passes through a refractive index change from high to low that cause the beam to bend in a first direction. Then, at the interface where the combined laser/visual beam 201/401 exits the window 801 and enters the cornea 3, i.e., interface between the concave surface 812 of the exit lens and the convex surface of the cornea, the beam passes through a refractive index change from low to high that cause the beam to bend in a second direction opposite the first direction.

The shape of the window 801 is chosen to be a meniscus lens. As such, the incidence angle of light has similar values on both surfaces 812, 813 of the window 801. The overall effect is that at the convex surface 813 the light bends away from the surface normal and at the concave surface 812 the light bends towards the surface normal. The effect is like when light passes through a plan parallel plate. Refraction on one surface of the plate is compensated by refraction on the other surface a light passing through the plate does not change its direction. Refraction at the entering, convex surface 712 of the exit lens 710 distal to the eye is minimized by setting the curvature of the entering surface such that angle of incidence β of light 201/401 at the entering surface is close to a surface normal 707 to the entering surface at the intersection point 708.

Here, the exit lens 710, the window 801, and the eye 1 are arranged as an axially symmetric system with a first optical axis 705. In practice, axial symmetry is an approximation because of manufacturing and alignment inaccuracies of the optical components, the natural deviation from symmetry of the eye and the inaccuracy of the alignment of the eye relative to the window 801 and the exit lens 710 in a clinical setting. But, for design and practical purposes the eye 1, the window 801, and the exit lens 710 are considered as an axially symmetric first optical subsystem 1001.

With continued reference to FIG. 10a, the second optical subsystem 1002 of the optical system 1010 is optically coupled to the first optical subsystem 1001 at an angle α relative to the first optical axis 705 of the first optical subsystem 1001. The advantage of this arrangement is that both optical subsystems 1001, 1002 can be designed at a much lower numerical aperture compared to a system where all optical components are designed on axis with a common optical axis.

The second optical subsystem 1002 includes the focusing objective 826 and various other components collectively indicated as an optical subsystem step 1003. Referring to FIG. 8b, these other components may include the scanner component 830 that optically couples with the laser source 200, and the visualization system 828, which may include a visual observation apparatus 400 and a telescope 842.

Returning to FIG. 10a, as previously described, the second optical subsystem 1002 includes mechanical parts (not shown) including a motorized first plate 844 and a motorized second plate 846 for moving one or more of the focusing objective 826 and the visualization system 828. The second optical subsystem 1002 may further include mechanical parts (not shown) configured to rotate the entire second optical subsystem around the first optical axis 705 of the first optical subsystem 1001. This allows optical access to the whole 360-degree circumference of the irido-corneal angle 13 of the eye 1.

With reference to FIG. 10b, flexibility in arranging the first and second optical subsystems 1001, 1002, relative to each other may be provided by an optical interface 1004 interposed between the optical output of the second optical subsystem 1002 and the optical input of the first optical subsystem 1001. In one embodiment, the optical interface 1004 may include one or more reflecting surfaces 740, prisms (not shown) or optical gratings (not shown) configured to receive the optical output, e.g., combined laser/visual beam 201/401, of the second optical subsystem 1002, change or adjust the direction of the combined laser/visual beam, and direct the beam to the optical input of the first optical subsystem 1001 while preserving the angle α between the first optical axis 705 and the second optical axis 706.

In another configuration, the optical interface 1004 further includes mechanical parts (not shown) configured to rotate 741 the optical interface of reflecting surfaces 740 around the first optical axis 705 of the first optical subsystem 1001, while keeping the second optical subsystem 1002 stationary. Accordingly, the second optical axis 706 of the second optical subsystem 1002 can be rotated around the first optical axis 705 of the first optical subsystem 1001. This allows optical access to the whole 360-degree circumference of the irido-corneal angle 13 of the eye 1.

With considerations described above with reference to FIGS. 9a, 9b and 9c, the design of the first optical subsystem 1001 is optimized for angled optical access at an angle α relative to the first optical axis 705 of the first optical subsystem 1001. Optical access at the angle α compensates for optical aberrations of the first optical subsystem 1001. Table 1 shows the result of the optimization at access angle α=72 degrees with Zemax optical design software package. This design is a practical embodiment for image guided femtosecond glaucoma surgery.

TABLE 1

| | | | | Center |
|---|---|---|---|---|
| | Structure and | Refractive | Radius | Thickness |
| Surface | Material | index | [mm] | [mm] |
| concave | exit lens 710 of | 1.45 | −10 | 4.5 |
| surface | focusing objective | | | |
| 711, | head 700. | | | |
| convex | fused silica | | | |
| surface | | | | |
| 712 | | | | |
| concave | window 801 of | 1.50 | −10.9 | 1.0 |
| surface | patient interface | | | |
| 812, | 800. | | | |
| convex | BK7G18 | | | |
| surface | | | | |
| 813 | | | | |
| 3 | cornea | 1.36 | −7.83 | 0.54 |
| 8 | aqueous humor | 1.32 | −6.53 | 3.5 |
| target | ophthalmic tissue | 1.38 | N/A | 0 to 1 mm |

This design produces diffraction limited focusing of 1030 nm wavelength laser beams with numerical aperture (NA) up to 0.2. In one design, the optical aberrations of the first optical subsystem 1001 are compensated to a degree that the Strehl ratio of the first optical subsystem for a laser beam 201 with numerical aperture larger than 0.15 at the irido-corneal angle is larger than 0.9. In another design, the optical aberrations of the first optical subsystem 1001 are partially compensated, the remaining uncompensated aberrations of the first optical system are compensated by optical elements of the second optical subsystem 1002 to a degree that the Strehl ratio of the combined first and second optical subsystem for laser beam 201 with numerical aperture larger than 0.15 at the irido-corneal angle is larger than 0.9.

Surgical Treatments Overview

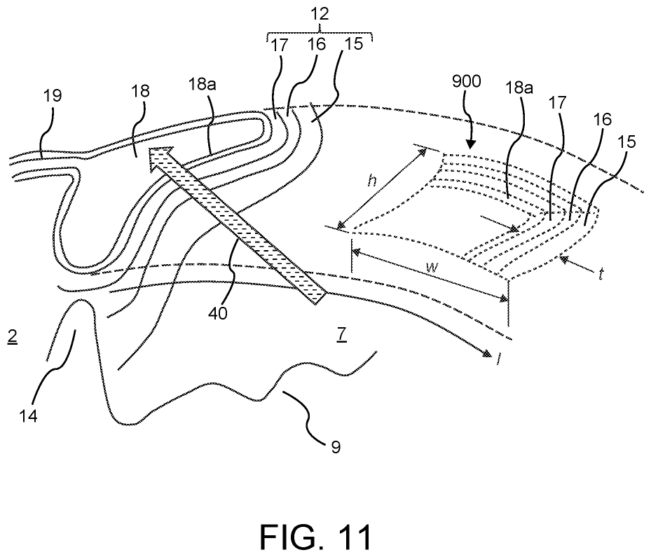

FIG. 11 is a three-dimensional schematic illustration of anatomical structures of the eye relevant to the surgical treatment enabled by the integrated surgical system 1000. To reduce the IOP, laser treatment targets ocular tissues that affect the trabecular outflow pathway 40. These ocular tissues may include the trabecular meshwork 12, the scleral spur 14, the Schlemm's canal 18, and the collector channels 19. The trabecular meshwork 12 has three layers, the uveal 15, the corneoscleral meshwork 16, and the juxtacanalicular tissue 17. These layers are porous and permeable to aqueous, with the uveal 15 being the most porous and permeable, followed by the corneoscleral meshwork 16. The least porous and least permeable layer of the trabecular meshwork 12 is the juxtacanalicular tissue 17. The inner wall 18a of the Schlemm's canal 18, which is also porous and permeable to aqueous, has characteristics similar to the juxtacanalicular tissue 17.

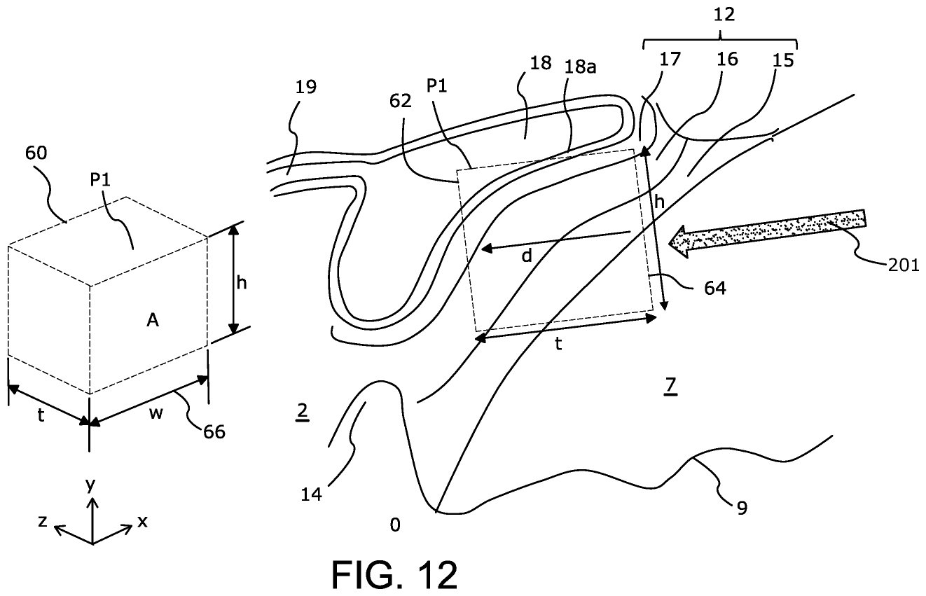

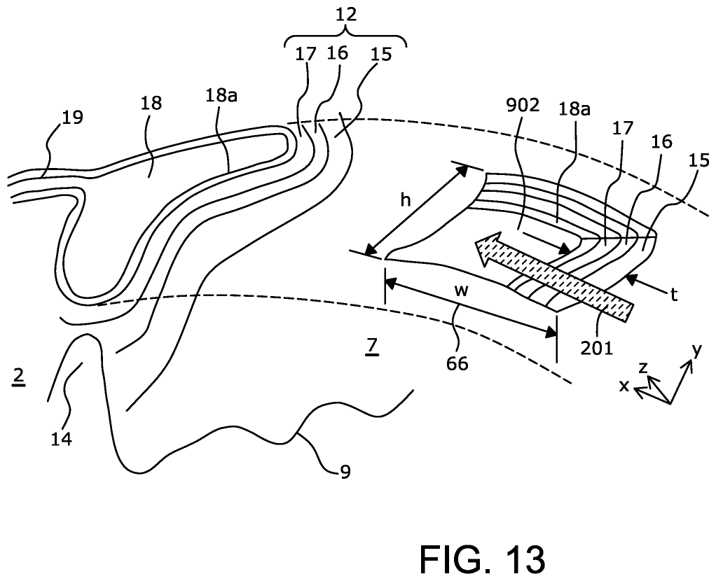

FIG. 12 includes three-dimensional illustrations of a treatment pattern P1 to be applied by the integrated surgical system 1000 to affect the surgical volume 900 of ocular tissue shown in FIG. 11, and a two-dimensional schematic illustration of the treatment pattern P1 overlaying anatomical structures to be treated. FIG. 13 is a three-dimensional schematic illustration of the anatomical structures of the eye including an opening 902 through the trabecular meshwork 12 that results from the application of the laser treatment pattern of FIG. 12. The opening 902 may also be referred to as a channel or aperture. The opening 902 provides and outflow pathway 40 that reduces the flow resistance in the ocular tissue to increase aqueous flow from the anterior chamber 7 into the Schlemm's canal 18 and thereby reduce the IOP of the eye.

Surgical treatments reduce outflow pathway resistance while minimizing ocular tissue modification through design and selection of laser treatment patterns. A treatment pattern is considered to define a collection of a laser-tissue interaction volumes, referred to herein as cells. The size of a cell is determined by the extent of the influence of the laser-tissue interaction. When the laser spots, or cells, are spaced close along a line, the laser creates a narrow, microscopic channel. A wider channel can be created by closely spacing a multitude of laser spots within the cross section of the channel. The arrangement of the cells may resemble the arrangement of atoms in a crystal structure.

With reference to FIG. 12, a treatment pattern P1 may be in the form of a cubic structure that encompasses individual cells arranged in regularly spaced rows, columns and sheets or layers. The treatment pattern P1 may be characterized by x, y, z dimensions, with x, y, z coordinates of the cells being calculated sequentially from neighbor to neighbor in the order of a column location (x coordinate), a row location (y coordinate), and a layer location (z coordinate). A treatment pattern P1 as such, defines a three-dimensional model of ocular tissue to be modified by a laser or a three-dimensional model of ocular fluid to be affected by a laser.

A treatment pattern P1 is typically defined by a set of surgical parameters. The surgical parameters may include one or more of a treatment area A that represents a surface area or layer of ocular tissue through which the laser will travel. The treatment area A is determined by the treatment height, h, and the lateral extent of the treatment, w. A treatment thickness t that represents the level to which the laser will cut into the ocular tissue from the distal extent or border of the treatment volume at or near Schlemm's canal 18 to the proximal extent or border at or near the surface of the trabecular meshwork 12. Thus, a laser applied in accordance with a treatment pattern may affect or produce a surgical volume that resembles the three-dimensional model of the treatment pattern or may affect fluid located in an interior of an eye structure resembled by the three-dimensional model.

Additional surgical parameters define the placement of the surgical volume or affected volume within the eye. For example, with reference to FIGS. 11 and 12, placement parameters may include one or more of a location l that represents where the treatment is to occur relative to the circumferential angle of the eye, and a treatment depth d that represents a position of the three-dimensional model of ocular tissue or ocular fluid within the eye relative to a reference eye structure. In the following, the treatment depth d is shown and described relative to the region where the anterior chamber 7 meets the trabecular meshwork 12. Together, the treatment pattern and the placement parameters define a treatment plan.

A femtosecond laser provides highly localized, non-thermal photo-disruptive laser-tissue interaction with minimal collateral damage to surrounding ocular tissue. Photo-disruptive interaction of the laser is utilized in optically transparent tissue. The principal mechanism of laser energy deposition into the ocular tissue is not by absorption but by a highly nonlinear multiphoton process. This process is effective only at the focus of the pulsed laser where the peak intensity is high. Regions where the beam is traversed but not at the focus are not affected by the laser. Therefore, the interaction region with the ocular tissue is highly localized both transversally and axially along the laser beam.

With reference to FIGS. 11 and 12, a surgical volume 900 of ocular tissue to be treated is identified by the integrated surgical system 1000 and a treatment pattern P1 corresponding to the surgical volume is designed by the integrated surgical system. Alternatively, the treatment pattern P1 may be designed first, and then an appropriate surgical volume 900 for applying the treatment pattern may be identified. The surgical volume 900 of ocular tissue may comprise portions of the trabecular meshwork 12 and the Schlemm's canal 18. For example, the surgical volume 900 of ocular tissue shown in FIG. 11 includes portions of the uveal 15, the corneoscleral meshwork 16, the juxtacanalicular tissue 17, and the inner wall 18a of the Schlemm's canal 18. The treatment pattern P1 defines a laser scanning procedure whereby a laser is focused at different depth locations in ocular tissue and then scanned in multiple directions to affect a three-dimensional volume of tissue comprising multiple sheets or layers of affected tissue.

With reference to FIGS. 12 and 13, during a laser treatment procedure, a surgical laser 201 may scan ocular tissue in accordance with the treatment pattern P1 to form an opening 902 that extends from the anterior chamber 7, through each of the uveal 15, the corneoscleral meshwork 16, the juxtacanalicular tissue 17 of the trabecular meshwork 12, and the inner wall 18a of the Schlemm's canal 18. While the example opening 902 in FIG. 13 is depicted as a continuous, single lumen defining a fluid pathway, the opening may be defined an arrangement of adjacent pores forming a sponge like structure defining a fluid pathway or a combination thereof. While the example opening 902 in FIG. 13 is in the shape of a cube, the opening may have other geometric shapes.

The movement of the laser as it scans to affect the surgical volume 900 follows the treatment pattern P1, which is defined by a set of surgical parameters that include a treatment area A and a thickness t. The treatment area A is defined by a width w and a height h. The width may be defined in terms of a measure around the circumferential angle. For example, the width w may be defined in terms of an angle, e.g., 90 degrees, around the circumferential angle.

Referring to FIGS. 11 and 12, an initial placement of the laser focus within the eye is defined by a set of placement parameters, including a depth d and a location l. The location l defines a point around the circumferential angle of the eye at which laser treatment will begin, while the depth d defines a point between the anterior chamber 7 and the Schlemm's canal 18 where the laser treatment begins or ends. The depth d is measured relative to the region where the anterior chamber 7 meets the trabecular meshwork 12. Thus, a first point that is closer to the Schlemm's canal 18 side of the trabecular meshwork 12 may be described as being deeper than a second point that is closer to the anterior chamber 7 side of the trabecular meshwork 12. Alternatively, the second point may be described as being shallower than the first point.

With reference to FIG. 13, the opening 902 resulting from laser application of the treatment pattern P1 resembles the surgical volume 900 and is characterized by an area A and thickness t similar to those of the surgical volume and the treatment pattern. The thickness t of the resulting opening 902 extends from the anterior chamber 7 and through the inner wall 18a of the Schlemm's canal 18, while the area A defines the cross-section size of the opening 902.

During a laser treatment procedure, a laser focus is moved to different depths d in ocular tissue and then scanned in two lateral dimensions or directions as defined by a treatment pattern P1 to affect a three-dimensional surgical volume 900 of ocular tissue comprising multiple sheets or layers of affected tissue. The two lateral dimensions are generally orthogonal to the axis of movement of the laser focus. With reference to FIG. 13, the movement of a laser focus during laser scanning is described herein with reference to x, y, and z directions or axes, wherein: 1) movement of the laser focus to different depths d through the thickness t of treatment pattern P1 or the surgical volume 900 of tissue corresponds to movement of the focus along the z axis, 2) movement of the laser focus in two dimensions or directions orthogonal to the z axis corresponds to movement of the laser focus along the width w of the treatment pattern P1 or the surgical volume 900 of tissue in the x direction, and movement of the laser focus along the height h of the treatment pattern P1 or the surgical volume 900 of tissue in the y direction.

As used herein scanning of the laser focus generally corresponds to a raster type movement of the laser focus in the x direction, the y direction, and the z direction. The laser focus may be located at a point in the z direction and then raster scanned in two dimensions or directions, in the x direction and the y direction. The focal point of the laser in the z direction may be referred to as a depth d within the treatment pattern P1 or the surgical volume 900 of tissue. The two-direction raster scanning of the laser focus defines a layer of laser scanning, which in turn produces a layer of laser-affected tissue.

During laser scanning, pulse shots of a laser are delivered to tissue within the volume of ocular tissue corresponding to the treatment pattern P1. Because the laser interaction volume is small, on the order of a few micrometers (m), the interaction of ocular tissue with each laser shot of a repetitive laser breaks down ocular tissue locally at the focus of the laser. Pulse duration of the laser for photo-disruptive interaction in ocular tissue can range from several femtoseconds to several nanoseconds and pulse energies from several nanojoules to tens of microjoules. The laser pulses at the focus, through multiphoton processes, breaks down chemical bonds in the molecules, locally photo-dissociate tissue material and create gas bubbles in wet tissue. The breakdown of tissue material and mechanical stress from bubble formation fragments the tissue and create clean continuous cuts when the laser pulses are laid down in proximity to one another along geometrical lines and surfaces.

Table 2 includes examples of treatment pattern parameters and surgical laser parameters for treating tissue. The range of the parameter set is limited by practical ranges for the repetition rate of the laser and the scanning speed of the scanners.

TABLE 2

| | Treatment | | | | | | |
|---|---|---|---|---|---|---|---|
| | pattern | | | | | | |
| | dimensions | Opening | Cell size | Laser | Laser | Laser | |
| | w[mm], | cross | w[μm], | average | repetition | pulse | Procedure |
| Tissue | h[mm], | section A | h[μm], | power | rate | energy | time |
| treated | t[mm] | [mm²] | t[μm] | [W] | [kHz] | [μJ] | [s] |
| Trabecular | 1.5, 0.2, 0.2 | 0.3 | 3, 3, 3 | 0.9 | 300 | 3 | 7.4 |
| meshwork | | | | | | | |
| Trabecular | 2, 0.2, 0.2 | 0.4 | 4, 4, 4 | 1 | 200 | 5 | 6.3 |
| meshwork | | | | | | | |
| Trabecular | 0.5, 0.2, 0.5 | 0.1 | 5, 5, 5 | 0.75 | 50 | 15 | 8.0 |
| meshwork | | | | | | | |
| Trabecular | 0.5, 0.2, 0.5 | 0.1 | 5, 5, 5 | 0.14 | 10 | 14 | 40.0 |
| meshwork | | | | | | | |
| Trabecular | 0.5, 0.2, 1.0 | 0.1 | 10, 10, 10 | 0.35 | 10 | 35 | 10.0 |
| meshwork | | | | | | | |
| Trabecular | 0.75, 0.25, | 0.1875 | 10, 10, 10 | 0.7 | 20 | 35 | 3.3 |
| meshwork | 0.35 | | | | | | |

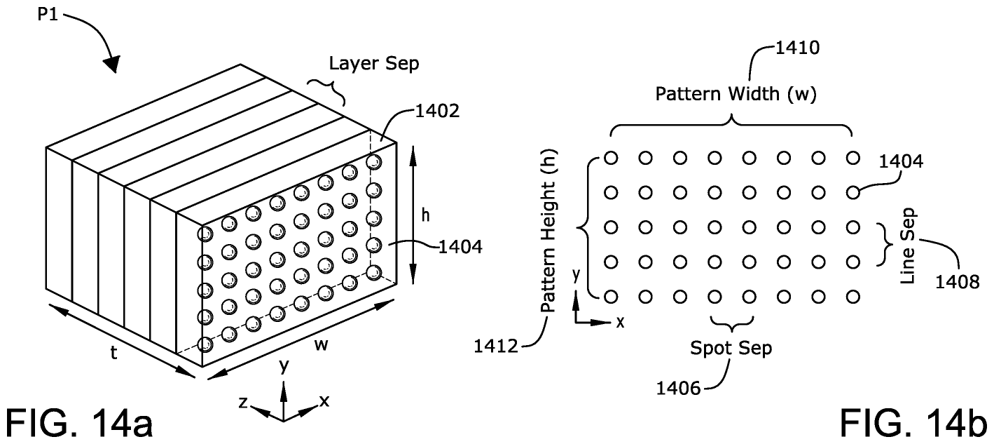

With reference to FIGS. 14a and 14b, a 3D treatment pattern P1 may be defined by a number of 2D treatment layers 1402 or treatment planes that are stacked to form a 3D treatment pattern characterized by a width w, height h, and depth or thickness t. Each individual treatment layer 1402 is in turn characterized by a pattern height h (equal to the height h of the 3D treatment pattern P1) and a pattern width w (equal to the width w of the 3D treatment pattern P1) and comprises an array of spots 1404 spaced apart to establish or fit within the height and width. The pattern width w corresponds to a distance along the circumference of the corneal angle parallel to the trabecular meshwork. This direction is also known as the circumferential direction. The pattern height h corresponds to a distance transverse to the circumference of the corneal angle perpendicular to the trabecular meshwork. This direction is also known as the azimuthal direction.

Each spot 1404 in the treatment pattern P1 corresponds to a site within a target volume of ocular tissue where optical energy is applied at a laser focus to create a micro-photodisruption site. With reference to FIG. 14b, each spot 1404 in a treatment layer 1402 is separated from a neighboring spot by programmable distances called spot separation (Spot Sep 1406) and a line separation (Line Sep 1408). A treatment layer 1402 is completed with the programmed pattern width w 1410 and pattern height h 1412 is achieved. Each treatment layer 1402 in the 3D treatment pattern P1 is separated from a neighboring layer by a layer separation (Layer Sep).

A treatment pattern P1 may be defined by a set of programmable parameters, such as shown in Table 3.

TABLE 3

| Parameter | Minimum | Maximum |
|---|---|---|
| width w | 10 μm | 2000 μm |
| height h | 10 μm | 2000 μm |
| depth/thickness t | 10 μm | 4000 μm |
| Spot Sep | 2 μm | 40 μm |
| Line Sep | 2 μm | 40 μm |
| Layer Sep | 2 μm | 200 μm |
| pulse energy | 0 μJ | 35 μJ |

Other, non-rectangular and more irregular treatment patterns can also be programmed and created in the tissue. These irregular patterns can still be decomposed to spots, lines, and layers and their extent characterized by width, height, and depth. Examples of irregular treatment patterns are described in U.S. patent application Ser. No. 16/838,858, entitled Method, System, and Apparatus for Generating Three-Dimensional Treatment Patterns for Laser Surgery of Glaucoma, the disclosure of which is hereby incorporated by reference.

In one example treatment pattern P1, the parameters are:
width=750 μm
height=250 μm
depth=350 μm
spot separation=10 μm
line separation=10 μm
layer separation=10 μm

During laser treatment, each treatment layer 1402 is individually created by scanning the laser focus in two dimensions, e.g., width and height, or z and y, to the various spots 1404 defining the layer, while the focus is fixed at the third dimension, e.g., depth or Z. Once a treatment layer 1402 is created, the focus is moved in the depth or z direction and the next treatment layer in the stack is created. This process is repeated until all treatment layers 1402 in the 3D treatment pattern P1 are created.

Laser Treatment Procedures with Fixed Image of Treatment Site

In accordance with embodiments disclosed herein, a fixed image of a treatment site is obtained and displayed by an integrated surgical system 1000 while scanning a focus of a laser beam at different depths of a treatment pattern. Following are descriptions of some laser treatment procedures that provide such fixed imaging.

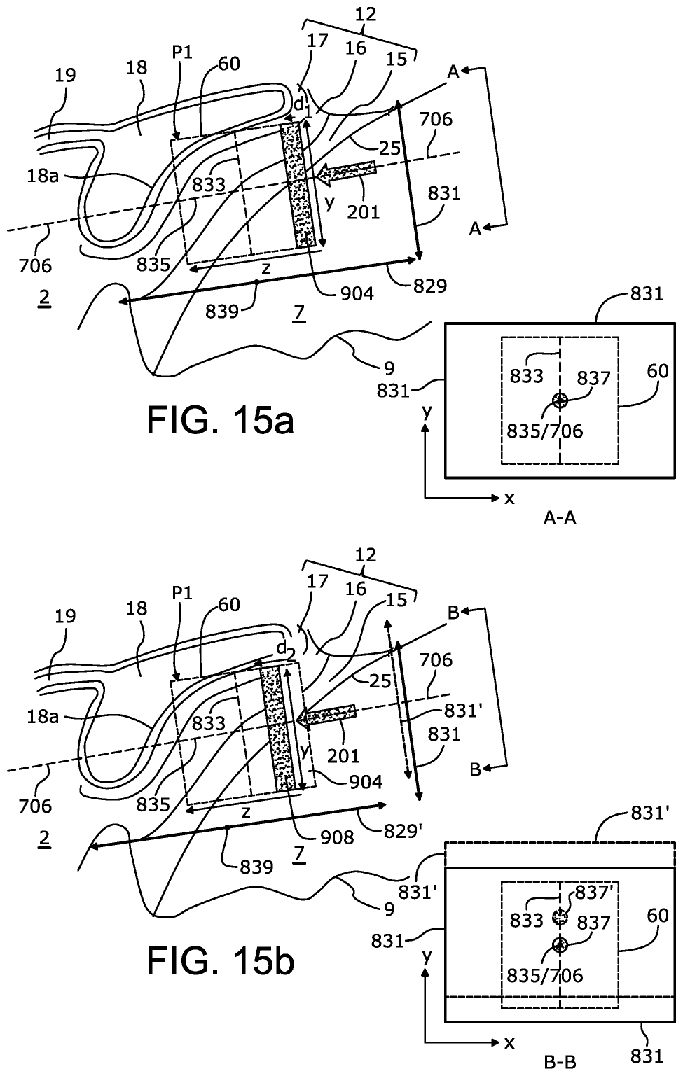

With reference to FIGS. 15a and 15b, in one type of laser treatment procedure the laser scanning of treatment layers begins at a shallow depth at the end of the treatment pattern P1 adjacent the anterior chamber 7 and proceeds, layer-by-layer, in a direction that generally corresponds to the direction of propagation of the laser beam 201. More specifically, and with reference to FIG. 15a, the laser scanning of treatment layers proceeds in the z direction (the depth direction) toward an anatomical structure, e.g., the Schlemm's canal 18, while the direction of propagation of the laser beam 201 also proceeds toward the same anatomical structure, e.g., the Schlemm's canal 18.

With reference to FIGS. 15a and 15b, in one type of laser treatment procedure the laser scanning of treatment layers begins at a shallow depth at the end of the treatment pattern P1 adjacent the anterior chamber 7 and proceeds, layer-by-layer, in a direction that generally corresponds to the direction of propagation of the laser beam 201. More specifically, and with reference to FIG. 15a, the laser scanning of treatment layers proceeds in the z direction (the depth direction) toward an anatomical structure, e.g., the Schlemm's canal 18, while the direction of propagation of the laser beam 201 also proceeds toward the same anatomical structure, e.g., the Schlemm's canal 18.

In FIG. 15a, the focus of the laser beam 201 may be initially located at a depth d₁ within a target volume 60 of ocular tissue. With reference to FIG. 8b, location of the focus of the laser beam 201 (also referred to herein at times, as the laser focus) at the depth d₁ may be accomplished through movement of the first plate 844 in a Z_{A} direction. Movement of the first plate 844 results in a corresponding movement of the focusing objective 826 and the focus of the laser beam 201 along the common optical axis 706 in the z direction of FIG. 15a. The depth d₁ places the laser focus in an initial layer 904 of tissue.

Continuing with FIG. 15a, an image of a treatment site 25 that includes the target volume 60 of ocular tissue is obtained by placing the depth of field 829 and the field of view 831 of the visualization system 828 at respective positions relative to the target volume of ocular tissue so that the target volume is within the depth of field and within the field of view. Placement of the depth of field 829 and the field of view 831 as such provides an initial or starting image of the treatment site 25 including the target volume 60 of ocular tissue.

The depth of field 829 of the visualization system 828 may be placed so a first axis 833 associated with the target volume 60 of ocular tissue is within the depth of field. The first axis 833 of the target volume 60 of ocular tissue may correspond to an axis that passes through an x-z plane of the target volume and extends along the height of the target volume in the y direction of FIG. 15a. In some embodiments, the first axis 833 passes through the center of the x-z plane and the depth of field 829 is placed so that the first axis 833 aligns with the center 839 of the depth of field.

The field of view 831 of the visualization system 828 may be placed so a point 837 of the field of view and a second axis 835 associated with the target volume 60 of ocular tissue are aligned with the common optical axis 706 of the first optical subsystem 1001. The point 837 of the field of view 831 may correspond to the center point of the x-y plane of the field of view. The second axis 835 of the target volume 60 of ocular tissue may correspond to an axis that passes through the center of the x-y plane of the target volume and extends along the length of the target volume in the z direction of FIG. 15a.

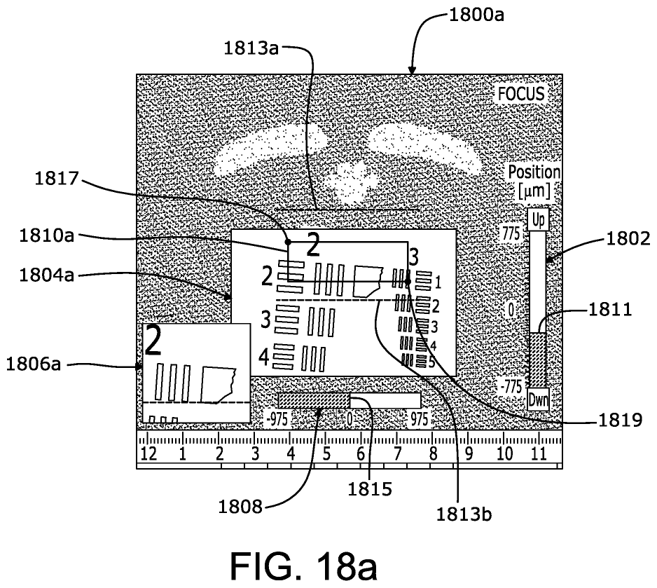

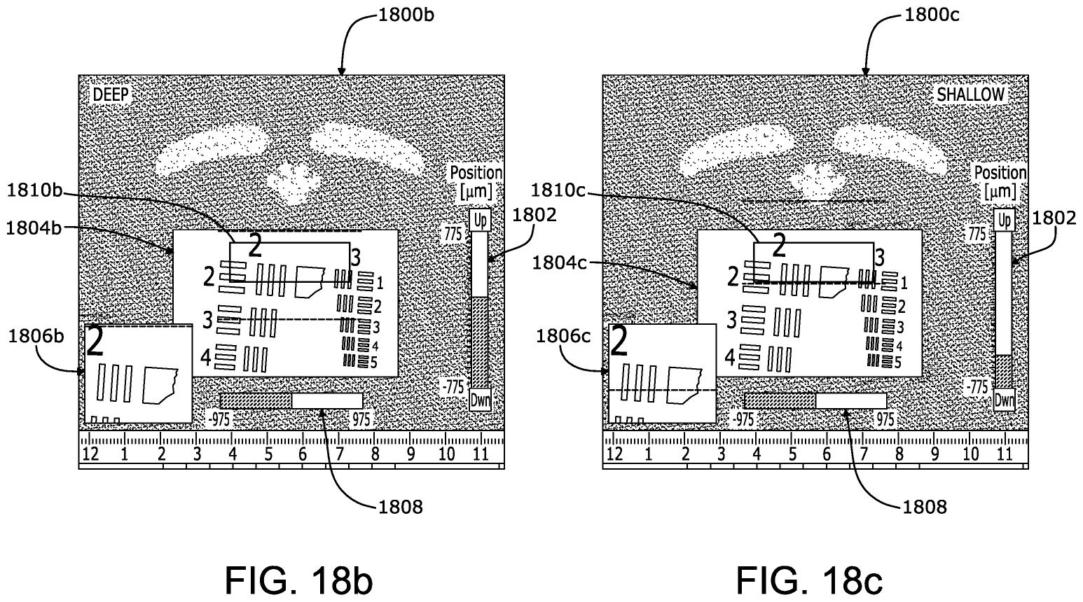

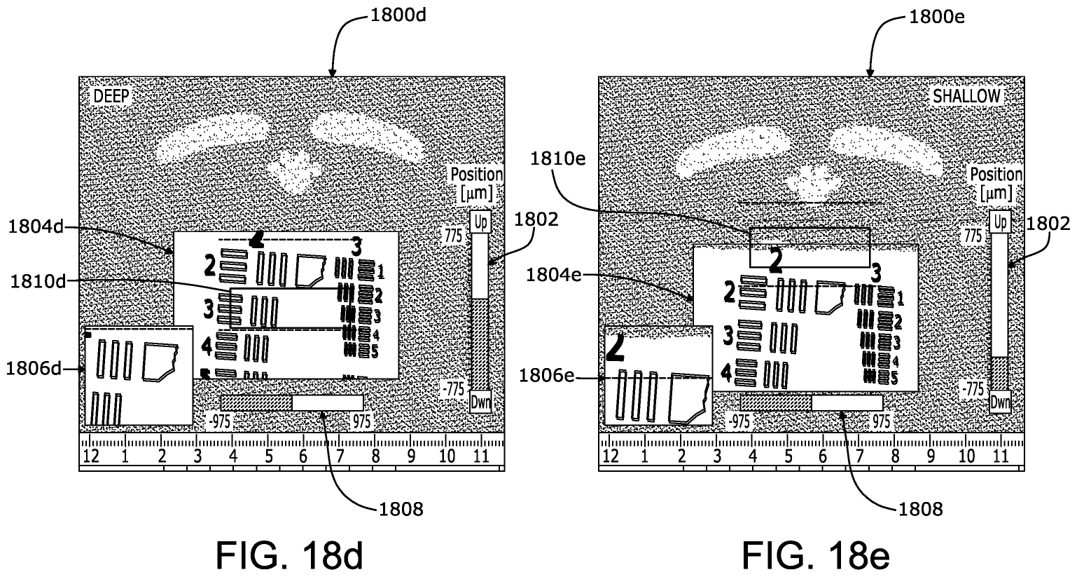

With the depth of field 829 and the field of view 831 placed as such, the visualization system 828 may obtain an initial image like the image shown in FIG. 18a. With reference to FIG. 8b, initial placement of the depth of field 829 and the field of view 831 may be accomplished through movement of one or both of the first plate 844 along the first mechanical axis 850 in a Z_{A} direction and the second plate 846 along the second mechanical axis 852 in the Z_{B} direction. Movement of these plates 844, 846 results in corresponding movements of the visualization system 828 and the focusing objective 826 and thus movements of the depth of field 829 and the field of view 831 relative to the target volume 60 of ocular tissue.

Continuing with FIG. 15a, with the depth of field 829 and the field of view 831 of visualization system 828 placed to capture an image of the treatment site 25 and the laser focus positioned at the initial depth d₁, the laser focus is scanned in multiple directions while being maintained at the initial depth. With reference to FIG. 14a, the multiple directions are the x direction (or circumferential direction) and the y direction (or azimuthal direction), where the x direction is into the plane of FIG. 15a.

With reference to FIG. 15b, the scanning of the laser focus of the laser beam 201 in the multiple directions results in the photodisruption of the initial layer 904 of tissue. The laser focus of the laser beam 201 is then moved along the common optical axis 706 in the z direction toward the Schlemm's canal 18 to another depth d₂. With reference to FIG. 8b, movement of the laser focus of the laser beam 201 is accomplished by movement of the first plate 844 along the first mechanical axis 850 in a Z_{A} direction which results in a corresponding movement of the focusing objective 826 and the focus of the laser beam 201 in the z direction of FIG. 15b. This depth d₂ places the laser focus at a subsequent layer 908 of tissue deeper than the initial layer 904. Once the laser focus is positioned at the subsequent layer 908, the laser focus is scanned in multiple directions while being maintained at that depth.

Typically, movement of the first plate 844 in a Z_{A} direction as just described also results in a corresponding movement of the visualization system 828 in a Z_{A} direction and corresponding movements or shifts of the depth of field 829 and the field of view 831 of the visualization system relative to the target volume 60 of ocular tissue. In FIG. 15b, the shifted depth of field due to this movement is indicated by callout 829′, while the shifted field of view is indicated by 831′. Regarding the position of the shifted depth of field 829′, it is shifted in the z direction relative to the first axis 833 associated with the target volume 60 of ocular tissue such that the first axis is not aligned with the center 839 of the shifted depth of field. This shift, however, does not affect the quality, e.g., focus, of the image captured by the visualization system 828 because the entirety of the target volume 60 along its depth in the z direction is still within the shifted depth of field 829′ of the visualization system.

Regarding the position of the shifted field of view 831′, it is offset in the y direction relative to the second axis 835 associated with the target volume 60 of ocular tissue. This shift results in a shifted center 837′ of the field of view that is not aligned with the second axis 835 of the target volume 60 (unlike that shown in FIG. 15a). This position of the shifted field of view 831′ relative to the target volume 60 of ocular tissue produces a corresponding shift in the field of view of the image captured by the visualization system 828. As described later below with reference to FIGS. 18a-18e, left unaccounted for, such movement of the field of view 831′ relative to the target volume 60 of ocular tissue causes the visualization system 828 to capture an image of the treatment site that has a different field of view relative to the initial image.

To avoid shifting and movement of the field of view during laser scanning, in accordance with embodiments disclosed herein, the field of view 831 of the visualization system 828 is maintained at its initial position relative to the target volume 60 of ocular tissue through independent movement of the visualization system 828. In one configuration, the independent movement of the visualization system 828 is in a direction opposite the z direction shown in FIG. 15b. For example, with reference to FIG. 8b, such independent movement of the visualization system 828 is accomplished by movement of the second plate 846 along the second mechanical axis 852 in a Z_{B} direction opposite the direction of movement of the first plate 844.

This movement of the second plate 846 results in a corresponding movement of the field of view of the visualization system 828 in the y direction shown in FIG. 15b and maintains the position of the field of view 831 relative to the target volume 60 of ocular tissue at its initial position. In FIG. 15b, maintenance of the position of the field of view 831 resulting from this movement of the visualization system 828 shows the field of view at a location corresponding to the initial location shown in FIG. 15a. Movement of the visualization system 828 as such maintains the position of the field of view relative to the target volume 60 of ocular tissue such that the point 837 of the field of view remains aligned with the second axis 835 of the target volume.

Returning to FIG. 15b, after scanning of the subsequent layer 908, the focus of the laser beam 201 is moved in the z direction toward the Schlemm's canal 18 to additional depths and scanned through additional treatment layers until all layers of the target volume 60 of ocular tissue have been treated. Each movement of the laser focus along the common optical axis 706 in the z direction is accompanied by a corresponding movement of the visualization system 828 as described above. As such, the position of the field of view 831 of the visualization system 828 is maintained at its initial position relative to the target volume 60 of ocular tissue and the field of view of the image of the treatment site 25 captured by the visualization system is maintained consistent with the initial or starting image of the treatment site.

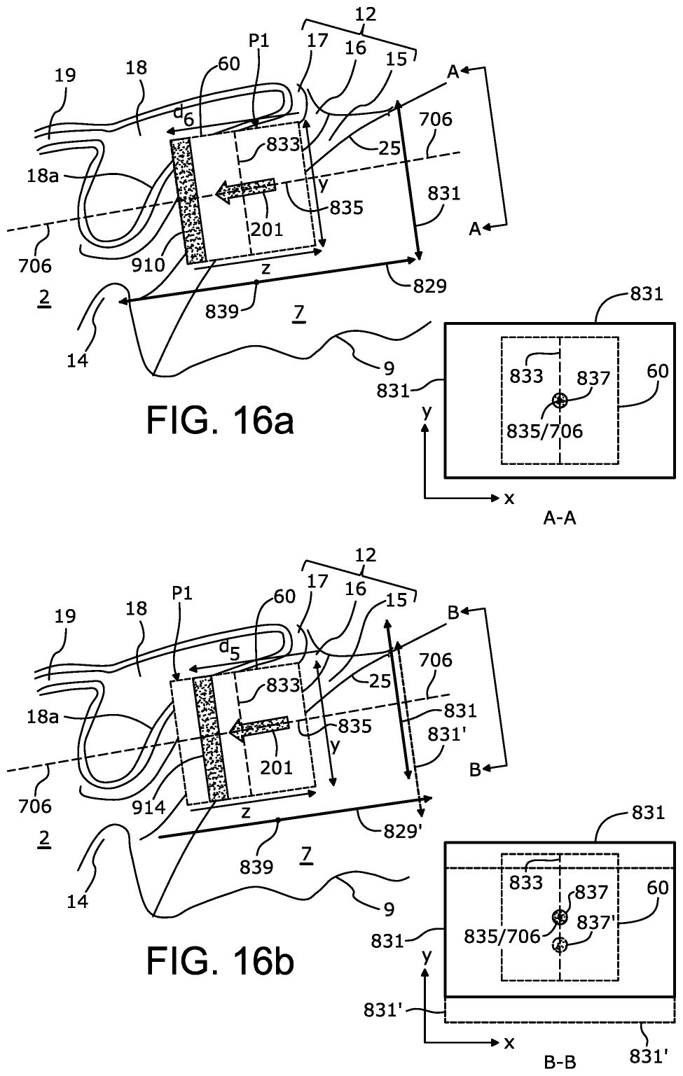

With reference to FIGS. 16a and 16b, in an alternative laser treatment procedure the laser scanning of treatment layers begins at a deep depth at the end of the treatment pattern P1 adjacent the Schlemm's canal 18 and proceeds, layer-by-layer, in a direction generally opposite to or against the direction of propagation of the laser beam 201. More specifically, and with reference to FIG. 16a, the laser scanning of treatment layers starts near an anatomical structure, e.g., the Schlemm's canal 18, and proceeds away from that structure in the z direction (the depth direction) toward the anterior chamber 7, while the direction of propagation of the laser beam 201 proceeds toward the structure.

In FIG. 16a, the focus of the laser beam 201 may be initially located at a depth d₆ within a target volume 60 of ocular tissue. With reference to FIG. 8b, location of the focus of the laser beam 201 (also referred to herein at times, as the laser focus) at the depth d₆ may be accomplished through movement of the first plate 844 in a Z_{A} direction. Movement of the first plate 844 results in a corresponding movement of the focusing objective 826 and the focus of the laser beam 201 along the common optical axis 706 in the z direction of FIG. 16a. The depth d₆ places the laser focus in an initial layer 910 of tissue.

Continuing with FIG. 16a, an image of a treatment site 25 that includes the target volume 60 of ocular tissue is obtained by placing the depth of field 829 and the field of view 831 of the visualization system 828 at respective positions relative to the target volume of ocular tissue so that the target volume is within the depth of field and within the field of view. Placement of the depth of field 829 and the field of view 831 as such provides an initial or starting image of the treatment site 25 including the target volume 60 of ocular tissue.

The depth of field 829 of the visualization system 828 may be placed so a first axis 833 associated with the target volume 60 of ocular tissue is within the depth of field. The first axis 833 of the target volume 60 of ocular tissue may correspond to an axis that passes through an x-z plane of the target volume and extends along the height of the target volume in the y direction of FIG. 16a. In some embodiments, the first axis 833 passes through the center of the x-z plane and the depth of field 829 is placed so that the first axis 833 aligns with the center 839 of the depth of field.

The field of view 831 of the visualization system 828 may be placed so a point 837 of the field of view and a second axis 835 associated with the target volume 60 of ocular tissue are aligned with the common optical axis 706 of the first optical subsystem 1001. The point 837 of the field of view 831 may correspond to the center point of the x-y plane of the field of view. The second axis 835 of the target volume 60 of ocular tissue may correspond to an axis that passes through the center of the x-y plane of the target volume and extends along the length of the target volume in the z direction of FIG. 16a.

With the depth of field 829 and the field of view 831 placed as such, the visualization system 828 may obtain an initial image like the image shown in FIG. 18a. With reference to FIG. 8b, initial placement of the depth of field 829 and the field of view 831 may be accomplished through movement of one or both of the first plate 844 along the first mechanical axis 850 in a Z_{A} direction and the second plate 846 along the second mechanical axis 852 in the Z_{B} direction. Movement of these plates 844, 846 results in corresponding movements of the visualization system 828 and the focusing objective 826 and thus movements of the depth of field 829 and the field of view 831 relative to the target volume 60 of ocular tissue.

Continuing with FIG. 16a, with the depth of field 829 and the field of view 831 of visualization system 828 placed to capture an image of the treatment site 25 and the laser focus positioned at the initial depth d₆, the laser focus is scanned in multiple directions while being maintained at the initial depth. With reference to FIG. 14a, the multiple directions are the x direction (or circumferential direction) and the y direction (or azimuthal direction), where the x direction is into the plane of FIG. 16a.

With reference to FIG. 16b, the scanning of the laser focus of the laser beam 201 in the multiple directions results in the photodisruption of the initial layer 910 of tissue. The laser focus of the laser beam 201 is then moved along the common optical axis 706 in the z direction toward the anterior chamber 7 to another depth d₅. With reference to FIG. 8b, movement of the laser focus of the laser beam 201 is accomplished by movement of the first plate 844 along the first mechanical axis 850 in a Z_{A} direction which results in a corresponding movement of the focusing objective 826 and the focus of the laser beam 201 in the z direction of FIG. 16b. This depth d₅ places the laser focus at a subsequent layer 914 of tissue deeper than the initial layer 904. Once the laser focus is positioned at the subsequent layer 914, the laser focus is scanned in multiple directions while being maintained at that depth.

Typically, movement of the first plate 844 in a Z_{A} direction as just described also results in a corresponding movement of the visualization system 828 in a Z_{A} direction and corresponding movements or shifts of the depth of field 829 and the field of view 831 of the visualization system relative to the target volume 60 of ocular tissue. In FIG. 16b, the shifted depth of field due to this movement is indicated by callout 829′, while the shifted field of view is indicated by 831′. Regarding the position of the shifted depth of field 829′, it is shifted in the z direction relative to the first axis 833 associated with the target volume 60 of ocular tissue such that the first axis is not aligned with the center 839 of the shifted depth of field. This shift, however, does not affect the quality, e.g., focus, of the image captured by the visualization system 828 because the entirety of the target volume 60 along its depth in the z direction is still within the shifted depth of field 829′ of the visualization system.

Regarding the position of the shifted field of view 831′, it is offset in the y direction relative to the second axis 835 associated with the target volume 60 of ocular tissue. This shift results in a shifted center 837′ of the field of view that is not aligned with the second axis 835 of the target volume 60 (unlike that shown in FIG. 16a). This position of the shifted field of view 831′ relative to the target volume 60 of ocular tissue produces a corresponding shift in the field of view of the image captured by the visualization system 828. As described later below with reference to FIGS. 18a-18e, left unaccounted for, such movement of the field of view 831′ relative to the target volume 60 of ocular tissue causes the visualization system 828 to capture an image of the treatment site that has a different field of view relative to the initial image.

To avoid shifting and movement of the field of view during laser scanning, in accordance with embodiments disclosed herein, the field of view 831 of the visualization system 828 is maintained at its initial position relative to the target volume 60 of ocular tissue through independent movement of the visualization system 828. In one configuration, the independent movement of the visualization system 828 is in a direction opposite the z direction shown in FIG. 16b. For example, with reference to FIG. 8b, such independent movement of the visualization system 828 is accomplished by movement of the second plate 846 along the second mechanical axis 852 in a Z_{B} direction opposite the direction of movement of the first plate 844.

This movement of the second plate 846 results in a corresponding movement of the field of view of the visualization system 828 in the y direction shown in FIG. 16b and maintains the position of the field of view 831 relative to the target volume 60 of ocular tissue at its initial position. In FIG. 16b, maintenance of the position of the field of view 831 resulting from this movement of the visualization system 828 shows the field of view at a location corresponding to the initial location shown in FIG. 16a. Movement of the visualization system 828 as such maintains the position of the field of view relative to the target volume 60 of ocular tissue such that the point 837 of the field of view remains aligned with the second axis 835 of the target volume.

Returning to FIG. 16b, after scanning of the subsequent layer 914, the focus of the laser beam 201 is moved in the z direction toward the anterior chamber 7 to additional depths and scanned through additional treatment layers until all layers of the target volume 60 of ocular tissue have been treated. Each movement of the laser focus along the common optical axis 706 in the z direction is accompanied by a corresponding movement of the visualization system 828 as described above. As such, the position of the field of view 831 of the visualization system 828 is maintained at its initial position relative to the target volume 60 of ocular tissue and the field of view of the image of the treatment site 25 captured by the visualization system is maintained consistent with the initial or starting image of the treatment site.

In another treatment, instead of creating a treatment pattern P1 one treatment layer 1402 at a time, the focus of the laser beam 201 is scanned in three dimensions. For example, while the laser focus is being moved transversely through a height and/or width, e.g., in the x and/or y direction, the laser focus is also oscillated back and forth axially through a depth, e.g., in the z direction. The treatment pattern P1 characterized by such scanning of the laser focus may be referred to as a “clearing pattern.” Oscillation of the laser focus through the depth in the z direction occurs simultaneous with transverse movement of the laser focus in the x and y directions. An example of scanning a laser in accordance with a clearing pattern is disclosed in U.S. patent application Ser. No. 17/202,257, the entire disclosure of which is hereby incorporated by reference.

In accordance with embodiments disclosed herein, to maintain a consistent quality, fixed field-of-view image of the treatment site while scanning a focus of a laser beam 201 at different depths of a treatment pattern or clearing pattern, the field of view 831 of the visualization system 828 is kept or maintained at its initial location through counter or offset movements of the visualization system. For example, for every movement of the focus of the laser beam 201 in a direction toward the Schlemm's canal 18 there is a corresponding movement of the visualization system 828 in the opposite direction toward the anterior chamber 7 that keeps or maintains the field of view 831 of the visualization system relative to the treatment site at its initial location. Likewise, for every movement of the focus of the laser beam 201 in a direction toward the anterior chamber 7 there is a corresponding movement of the focus of the visualization system 828 in the opposite direction toward the Schlemm's canal 18 that keeps or maintains the field of view 831 of the visualization system at its initial location.

While the foregoing descriptions of the imaging operation of the integrated surgical system 1000 may speak in terms of obtaining a starting or initial image and obtaining subsequent images, such terminology is used to aid in describing a sequential operation. In some embodiments, the visualization system 828 of the integrated surgical system 1000 is configured to obtain a continuous, real-time image of the relevant area of eye, and thus instead of there being a separate initial image followed by one or more subsequent images, there is one continuous image overtime. Furthermore, while the directions of movement among the physical structures, including the visualization system 828 and the focusing objective 826, are described as being opposite each other, other relative movements are possible depending on the geometry, i.e., optical axis arrangement of these structures.

Method of Fixed Imaging During Laser Treatment

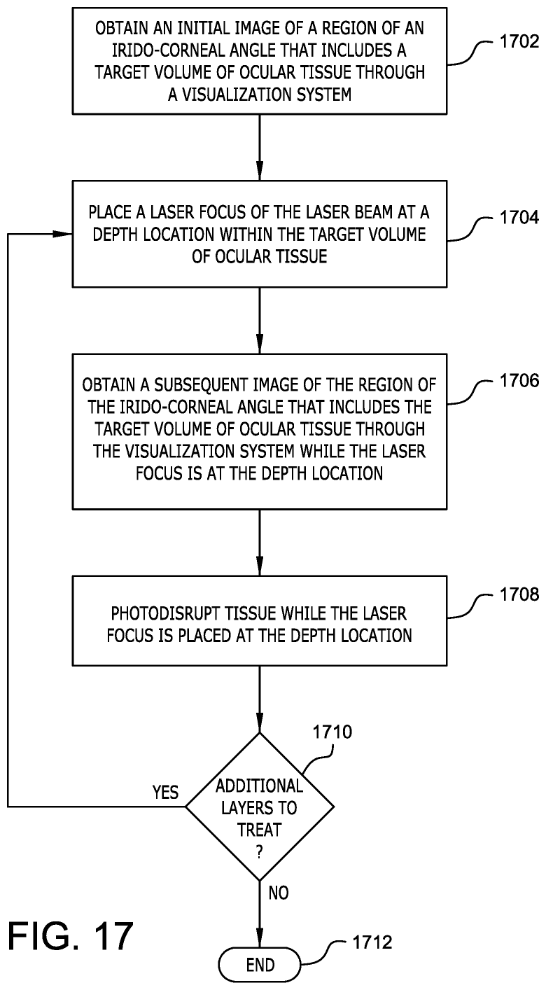

FIG. 17 is a flowchart of a method of imaging a region of an eye including a target volume of ocular tissue. The region of the eye may be, for example, the irido-corneal angle. The method may be performed during a laser treatment of the target volume to provide real-time imaging of the region of the eye. With reference to FIGS. 12, the target volume 60 of ocular tissue is characterized by a distal extent 62, a proximal extent 64, and a lateral extent 66. The distal extent 62 corresponds to the part or point of the target volume 60 that is most distal along the direction of propagation of the laser beam 201. The proximal extent 64 corresponds to the part or point of the target volume 60 that is most proximal along the direction of propagation of the laser beam 201. The lateral extent 66 corresponds to the distance or width w of the target volume 60 along the circumference angle.

The method, which may be performed by the integrated surgical system 1000 of FIG. 8b, begins at a point in a surgical procedure where the region of the eye, e.g., the irido-corneal angle, is accessible and the target volume 60 of ocular tissue has been identified for treatment. Systems and methods for accessing the irido-corneal angle are described in U.S. patent application Ser. No. 16/036,883, entitled Integrated Surgical System and Method for Treatment in the Irido-Corneal Angle of the Eye, the disclosure of which is hereby incorporated by reference. Systems and method for identifying volumes of ocular tissue for treatment and designing treatment patterns reference are described in U.S. patent application Ser. No. 16/125,588, entitled Non-Invasive and Minimally Invasive Laser Surgery for the Reduction of Intraocular Pressure in the Eye, the disclosure of which is hereby incorporated by reference.

With reference to FIG. 8b, the visualization system 828, e.g., a gonioscopic video camera, is configured to provide a direct image of the irido-corneal angle 13 of the eye during a laser treatment procedure. The direct image may be provided by the visualization system 828 to the control system 100, which in turn presents the image on a display 110 of the integrated surgical system. The control system 100 may be configured to add surgical guidance objects to the image to assist a surgeon in identifying and marking a treatable surgical area and a location of the target volume 60 of ocular tissue within the treatable surgical area.

FIGS. 18a-18c are schematic representations of images that may be obtained and displayed by the integrated surgical system 1000 of FIG. 8b with the visualization system 828 configured in accordance with embodiments disclosed herein. FIGS. 18d and 18e are schematic representations of images that would otherwise be obtained by a surgical system that is not configured in accordance with embodiments disclosed herein. The image inside the respective bounding box of each of FIGS. 18a-18e is a schematic representations of a gonioscopic image 1800a-1800e obtained by a visualization system 828. The gonioscopic image 1800a-1800e is of the same region of the irido-corneal angle of an eye. A vertical scale 1802 in micrometers (μm) may represent the height of the treatment pattern in the y direction (or azimuthal direction). The major sub-box 1804a-1804e corresponds to a region of the gonioscopic image 1800a-1800e, wherein the region is shown as a focus chart. For purposes of clarity in illustrating differences among the images, focus charts are shown instead of schematic images of ocular tissue.

Continuing with FIGS. 18a-18e, the minor sub-box 1806a-1806e represents a magnified portion of the images within the major sub-boxes 1804a-1804e. Changes in field of view and focus and other characteristics of the gonioscopic image 1800a-1800e are more discernable in the minor sub-boxes 1806a-1806e. A horizontal scale 1808 in micrometers (μm) represents the width of the treatment pattern along the circumferential angle and in the x direction (or circumferential direction). Also included in FIGS. 18a-18e are surgical guidance objects including a treatment overlay 1810a-1810e that represents a surgical treatment pattern, and a pair of horizontal bars that indicate a treatable surgical area of the laser surgical system. The treatment overlay 1810a-1810e, also referred to herein as treatment box, physically represents the location and size of a laser cut. These guidance objects are software constructs generated by the control system 100 and are added to the display of the gonioscopic images 1800a-1800e.

Returning to FIG. 17, at block 1702, and with additional reference to FIGS. 8b, 12, and 18a, the integrated surgical system 1000 obtains an initial image in the direction of the second optical axis 706 of a region of an irido-corneal angle 13 that includes a target volume 60 of ocular tissue through a visualization system 828. As shown in the embodiment of FIG. 8b, the visualization system 828 is optically coupled to a focusing objective 826 that, in turn is optically coupled to obtain a laser beam 201 from a laser source 200. With reference to FIG. 12, the initial image may be of the anterior chamber 7 and the trabecular meshwork 12 near the proximal extent 64 of the target volume 60 of ocular tissue. The initial image is displayed on the display 110 of the integrated surgical system 1000. With reference to FIG. 18a, the initial image may correspond to the gonioscopic image 1800a.

With additional reference to FIGS. 8b and 15a, the initial image 1800a of a region of an irido-corneal angle may be obtained by placing the depth of field 829 and the field of view 831 of the visualization system 828 at initial positions relative to the target volume 60 of ocular tissue such that the target volume is within the depth of field and the field of view. Such initial placement of the depth of field 829 and the field of view 831 of the visualization system 828 is typically done at the start of a surgical procedure after the patient's eye is docked to a patient interface 800 and may be done by moving the visualization system 828 and the focusing objective 826 an initial distance in an initial direction along a first mechanical axis 850. The first mechanical axis 850 is axially aligned with the front-end objective 836 of the focusing objective 826 and reflects off the reflecting mirror 740 to align with the second optical axis 706. The initial direction may be in either of two opposite directions as represented by the Z_{A} double arrow in FIGS. 8b and 8c.

With reference to FIGS. 15a, 15b, 16a, and 16b, the depth of field 829 and field of view 831 of the visualization system 828 may be initially placed such that the first axis 833 through the target volume 60 of ocular tissue in the y direction is aligned with the center 839 of the depth of field, and the second axis 835 through the target volume in the z direction is aligned with the center of the field of view. The initial image 1800a obtained after such initial placement the depth of field 829 and field of view 831 of the visualization system 828 has one or more image characteristics. These image characteristics may include, for example, field of view and one or more visual characteristics, such as quality of focus, magnification, resolution, contrast, and other quality factors (e.g., such as distortion and optical aberrations). Focus may be characterized in terms of sharpness, clarity, etc. Field of view may be characterized as the portion of an image that falls within a boundary.

In some embodiments, during initial placement of the depth of field 829 and field of view 831 relative to the target volume 60, the visualization system 828 and the focusing objective 826 are simultaneously or synchronously moved in the first direction along the first mechanical axis 850. For example, in the configuration of FIG. 8b the visualization system 828 and the focusing objective 826 are associated with the same first movement mechanism, e.g., first plate 844 and first motor 845. As such, movement of the first plate 844 results in a simultaneous or synchronous movement of the visualization system 828 and the focusing objective 826. In other embodiments, the visualization system 828 and the focusing objective 826 may be separately or asynchronously moved in the first direction along the first mechanical axis 850. For example, the visualization system 828 and the focusing objective 826 may be associated with independent movement mechanisms, each arranged to move its respective component the initial distance in the initial direction along the first mechanical axis 850. In this embodiment, movement of the visualization system 828 and the focusing objective 826 may be synchronous or asynchronous.

At block 1704, the integrated surgical system 1000 places a laser focus of the laser beam 201 at a depth location within the target volume of ocular tissue in accordance with the treatment pattern P1. To this end, the focusing objective 826 may be moved a first distance in a first direction along the first mechanical axis 850. In the embodiment of FIGS. 8b and 8c, the first direction of movement of the focusing objective 826 may be in either of two opposite directions as represented by the Z_{A} double arrow. More specifically for purposes of example only, with reference to FIG. 8c the focusing objective 826 may be moved a first distance 860 in a first direction 862 along the first mechanical axis 850.

With continued reference to FIG. 8c and additional reference to FIG. 15a, the focus of the laser beam 201 may be moved a first distance 860 to place it at an initial depth d₁ corresponding to the proximal extent 64 of the target volume 60 of ocular tissue. Alternatively, with continued reference to FIG. 8c and additional reference to FIG. 16a, the focus of the laser beam 201 may be moved a first distance 861 to place it at an initial depth d₆ corresponding to the distal extent 62 of the target volume 60 of ocular tissue. The first distance 860, 861 may be an increment corresponding to the distance the focus of the laser beam 201 is moved in the z direction during a scanning. For example, as described above with reference to FIGS. 14a and 14b, a laser focus may move in the z direction a distance corresponding to a layer separation, which for example may be 10 μm.

At block 1706, and with additional reference to FIGS. 8b, 18b and 18c, the integrated surgical system 1000 obtains a subsequent image 1800b, 1800c of the region of the irido-corneal angle through the visualization system 828 while the laser focus is at the depth location. The subsequent image is displayed on the display 110 of the integrated surgical system 1000. The subsequent image may correspond to a gonioscopic image 1800b, 1800c. As shown in FIGS. 18b and 18c, the subsequent image 1800b, 1800c has a field of view consistent with the initial image 1800a of FIG. 18a, and visual characteristics, e.g., focus, consistent with the initial image 1800a.

Details on the configuration and operation of the integrated surgical system 1000 that enable the obtaining of subsequent images having consistent field of view and consistent focus are provided after the description of the method of FIG. 17. Briefly for now and regarding field of view, the visualization system 828 is moved a second distance in a second direction along a second mechanical axis 852 to counter any movement of the visualization system 828 a first distance in a first direction along the first mechanical axis 850 that occurs during placement of the laser focus in block 1704. This second movement keeps or maintains the field of view 831 of the visualization system 828 at its initial position relative to the target volume 60 of ocular tissue.

At block 1708, the integrated surgical system 1000 may photodisrupt tissue while the focus of the laser beam 201 is placed at the depth location. For example, with reference to FIG. 15a, the integrated surgical system 1000 focuses light from a femtosecond laser 201 at a spot in the tissue at the shallow depth d₁ and applies optical energy to the tissue, which energy is at a level sufficient to photodisrupt the tissue. In another example, with reference to FIG. 16a, the integrated surgical system 1000 focuses light from a femtosecond laser 201 at a spot in the tissue at the deep depth d₆ and applies optical energy to the tissue, which energy is at a level sufficient to photodisrupt the tissue. In either case, optical energy is applied while scanning the laser beam 201 in multiple directions defining an initial layer 904 or treatment plane at the shallow depth d₁ (in FIG. 15a) or an initial layer 910 or treatment plane at the deep depth d₆ (in FIG. 16a) to thereby photodisrupt an initial layer of tissue of the target volume of ocular tissue. With reference to FIG. 13, the scanning may be in the form of a raster scan where the laser is scanned in a first direction along the lateral extent 66, i.e., the x direction, and then slightly repositioned in a second direction. i.e., the y direction, and then scanned again along the lateral extent.

At block 1710, the integrated surgical system 1000 determines if additional layers of the target volume 60 of ocular tissue are to be treated. If no additional layers are to be treated, the integrated surgical system ends the procedure at block 1712.

Returning to block 1710, if additional layers of the target volume 60 of ocular tissue are to be treated, the integrated surgical system 1000 repeats the placing (block 1704) and the capturing (block 1706) and the photodisrupting (block 1708) for one or more different depth locations within the target volume of ocular tissue.

The foregoing description of the method of FIG. 17 is based on the arrangement of the visualization system 828 and the focusing objective 826 illustrated in FIG. 8b, where the direction of movement of the visualization system by itself along the second mechanical axis 852 is described as opposite the direction of movement of the visualization system together with the focusing objection along the first mechanical axis 850. In a configuration where the visualization system 828 and the focusing objective 826 are not mechanically coupled together and instead move independently of each other, a way to describe relative movement of the visualization system 828 and the focusing objective 826 may be in terms of a common component, such as the dichroic mirror 834. Under this convention, each of the visualization system 828 and the front-end objective 836 of the focusing objective 826 may be described as moving toward or away from the dichroic mirror 834. However, it is entirely feasible that the relationship could be different, with both moving towards the dichroic. The nature of this relationship may depend upon the optical design of the system (e.g., number of lenses, magnification less than or greater than 1, the geometry of the design, etc.).

Fixed Field of View

As mentioned above, the integrated surgical system 1000 is configured to provide an image of the treatment site that has a consistent field of view during laser scanning. Consistent field of view means the portion of the subsequent image 1800b, 1800c that falls within the bounding box of FIG. 18b or 18c is the same as the portion of the initial image 1800a that falls within the bounding box of FIG. 18a. Perceptually, this means the image on the display 110 of the integrated surgical system 1000 appears still, without any shifting, during laser scanning.

In accordance with embodiments disclosed herein, images having a consistent field of view are obtained by moving the visualization system 828 in a way that keeps or maintains the field of view 831 of the visualization system at its initial position relative to the target volume 60 regardless of movement of the laser focus to different depths during laser scanning. In doing so, the field of view of the image of the target volume 60 obtained by the visualization system 828 and displayed by the integrated surgical system 1000 is fixed.

In one possible configuration, the field of view 831 is kept or maintained at its initial position relative to the target volume 60 by returning the field of view to its initial position after movement of the field of view. In this configuration, the visualization system 828 may first be moved together with the focusing objective 826 a first distance in a first direction along the first mechanical axis 850, and then quickly moved a second distance in a second direction along a second mechanical axis 852 without corresponding movement by the focusing objective along the second mechanical axis. The first movement of the visualization system 828 moves the field of view 831 away from its initial position. The second movement, however, returns the field of view 831 of the visualization system 828 back to its initial position

In another possible configuration, the field of view 831 is kept or maintained at its initial position relative to the target volume 60 by preventing movement of the field of view in the first place. In this configuration, a movement of the visualization system a first distance in a first direction along the first mechanical axis 850 that would move the field of view from its initial position is countered by a simultaneous movement of the visualization system a second distance in a second direction along a second mechanical axis 852 without corresponding movement by the focusing objective 826 along the second mechanical axis. This simultaneous counter movement along the second mechanical axis 852, in effect, keeps or maintains the field of view 831 of the visualization system 828 at its initial position relative to the target volume 60. In other words, the field of view 831 does not move from its initial position.

With reference to the arrangement of FIGS. 8b and 8c, the first direction of movement of the visualization system 828 may be in either of two opposite directions as represented by the Z_{A} double arrow. More specifically for purposes of example only, with reference to FIG. 8c the visualization system 828 may be moved a first distance 860 in a first direction 862 along the first mechanical axis 850. In the integrated surgical system 1000 of FIG. 8b, this first movement of the visualization system 828 is simultaneous or synchronous with the movement of the focusing objective 826 due to the each of the visualization system and focusing objective being associated with the same first movement mechanism, e.g., first plate 844 and first motor 845.

Regarding the opto-mechanics of this second movement of the visualization system 828, as shown in FIGS. 8b and 8c the second mechanical axis 852 is axially aligned with the telescope 842 of the visualization system 828 and is input to the dichroic mirror 843 of the focusing objective 826 to align with the first mechanical axis 850. In the arrangement of FIGS. 8b and 8c, the second movement of the visualization system 828 in the second direction may be in either of two opposite directions as represented by the Z_{B} double arrow. More specifically for purposes of example only, with reference to FIG. 8c the visualization system 828 may be moved a second distance 864 in a second direction 866 along the second mechanical axis 852. The focusing objective 826, however, is not moved in the second direction. This movement of the visualization system 828 a second distance in a second direction along a second mechanical axis 852 keeps or maintains the field of view 831 of the visualization system 828 at its initial position and thus results in a subsequent image 1800b, 1800c of the region of the irido-corneal angle that has a field of view consistent with the initial image 1800a of FIG. 18a.

Considering further the opto-mechanical arrangement of FIG. 8b, any movement of the visualization system 828 in a first direction (e.g., either of two opposite directions as represented by the Z_{A} double arrow in FIG. 8b) may be accounted for by a movement of the visualization system in a second direction (e.g., either of two opposite directions as represented by the Z_{B} double arrow in FIG. 8b) without corresponding movement of the focusing objective 826. More specifically for purposes of example only, with reference to FIG. 8b, any movement of the first plate 844 that moves the visualization system 828 together with the focusing objective 826 is countered by movement of the second plate 846 that moves only the visualization system.

This movement of the second plate 846 keeps or maintains the field of view of the visualization system 828 at the initial position and thus results in a subsequent image 1800b, 1800c of the region of the irido-corneal angle that has a field of view consistent with the initial image 1800a of FIG. 18a. As noted above, in some embodiments movement of the visualization system 828 in the second direction along the second mechanical axis 852 is simultaneous or synchronous with movement of the focusing objective 826 in the first direction along the first mechanical axis 850 (at block 1704). In other embodiments movement of the visualization system 828 in the second direction along the second mechanical axis 852 is separate from, or asynchronous with, movement of the focusing objective 826 in the first direction along the first mechanical axis 850 (at block 1704).

In either of simultaneous/synchronous or separate/asynchronous movement, in some embodiments the second distance by which the visualization system 828 is moved (at block 1706) in a second direction along the second mechanical axis 852 without corresponding movement of the focusing objective 826 is a transfer function of the first distance by which each of the focusing objective 826 and the visualization system 828 are moved (at block 1704) in a first direction along the first mechanical axis 850. With reference to FIG. 8c, in one configuration, this transfer function is simply a scalar value, such that the second distance 864 the visualization system 828 moves in the second direction 866 along the second mechanical axis 852 is a constant value, which is linearly proportional to the first distance 860 the focusing objective 826 (together with the visualization system 828) moves in the first direction 862 along the first mechanical axis 850.

In FIG. 8c, the transfer function is one in which the first distance 860 and the second distance 864 are equal. The two distances 860, 864, however, may not be equal. For example, in some configurations, the second distance 864 is less than the first distance 860. In other words, the ratio of second distance/first distance is less than one (1.0) and may range, for example, from 0.2 to 0.6. Numerous other ratios outside of this example range are possible, as the ratio value is a function of characteristics of the visualization system 828, such as magnification, and geometry, i.e., the angle between the first mechanical axis 850 of the focusing objective 826 and the second mechanical axis 852 of the visualization system.

With reference to FIG. 8b, in some embodiments, the control system 100 is configured to control operation of the second movement mechanism, e.g., the second plate 846 and the camera motor 847, based on the operation of the first movement mechanism, e.g., the first plate 844 and the objective motor 845, so that movement of the camera motor (and thus movement of the visualization system 828) is triggered by movement of the objective motor (and thus movement of the focusing objective 826). To this end, the control system 100 may be programmed to detect activation of the objective motor 845 and movement of the first plate 844 in a first direction 862 along the first mechanical axis 850 and to immediately activate the camera motor 847 and initiate a corresponding movement of the second plate 846 in a second direction 866 along the second mechanical axis 852. Note, activation of the objective motor 845 and movement of the first plate 844 may be automatically controlled by the control system 100 in accordance with a scanning treatment pattern or activation may be operator initiated. The control system 100 may be further programmed to detect deactivation of the objective motor 845 and a stoppage of movement of the first plate 844 and to immediately deactivate the camera motor 847 to stop a corresponding movement of the second plate 846 in the second direction 866.

Fixed Focus

As mentioned above, the integrated surgical system 1000 is configured to provide an image of the treatment site that has a consistent focus during laser scanning. Consistent focus means the sharpness or clarity of the subsequent image 1800b, 1800c is the same as those of the initial image 1800a. Quantitatively, this consistency in focus may be evidenced using a contrast-based spatial resolution approach. This is an industry standard, involving groups of line pairs (black and white) whose separation between lines gets successively smaller. The resolution limit is the line pair size at which one cannot distinguish between two black lines. The classic layout is the United States Air Force 1951 resolution target. Focus is a measure of resolution: when the image is de-focused then the resolution has degraded. Experimental techniques to assess “best focus” typically involve moving an Air Force target in the imaging direction to see it go in and out of focus.

In accordance with embodiments disclosed herein, images having a consistent focus are obtained by configuring the visualization system 828 with a long depth of field relative to a dimension of the target volume 60 of ocular tissue. For example, the depth of field 829 of the visualization system 828 may be 2 to 10 times greater than the thickness of the target volume 60, which from Table 3 above may range from 10 μm to 4000 μm. As such, any movement of the depth of field 829 that results from movement of the visualization system 828 does not cause the target volume 60 to fall outside the depth of field. Accordingly, the focus of the subsequent image 1800b, 1800c obtained by the visualization system 828 and displayed by the integrated surgical system 1000 is consistent with that of the initial image 1800a. Perceptually, this means the image on the display 110 of the integrated surgical system 1000 does not appear to change focus during laser scanning.

Comparison of Display Images

With reference to FIGS. 15a and 18b, a subsequent image 1800b of the region of an irido-corneal angle may be obtained, for example, by moving the visualization system 828 and the focusing objective 826 as described above with reference to blocks 1704 and 1706 of FIG. 17 to place the focus of a laser beam 201 at a shallow depth d₁ (referred to herein as a “shallow position”), while the field of view 831 of the visualization system 828 is kept or maintained at its initial position relative to the target volume 60 of ocular tissue, and the target volume remains within the depth of field 829 of the visualization system.

As described above, the field of view 831 of the visualization system 828 may be kept or maintained at its initial position through movement of the visualization system that returns the field of view to its initial position after movement of the field of view, or that prevents movement of the field of view. Also, as described above, the target volume remains within the depth of field 829 of the visualization system 828 due to the long depth of field.

In comparing the initial image 1800a (shown in FIG. 18a) and the subsequent image 1800b (shown in FIG. 18b) it is noted that the respective images have the same or substantially the same focus characteristic and field of view. For example, the images inside of major sub-box 1804b and minor sub-box 1806b of FIG. 18b are consistent with the images inside the major sub-box 1804a and minor sub-box 1806a of FIG. 18a. Furthermore, the position of the treatment overlay 1810b of FIG. 18b is consistent with the position of the treatment overlay 1810a of FIG. 18a.

With reference to FIGS. 16a and 18c, a subsequent image 1800c of the region of an irido-corneal angle may be obtained by moving the visualization system 828 and the focusing objective 826 as described above with reference to blocks 1704 and 1706 of FIG. 17 to place the laser focus at a deep depth d₆ (referred to herein as a “deep position.”), while the field of view of the visualization system 828 is kept or maintained at its initial position relative to the target volume 60 of ocular tissue, and the target volume remains within the depth of field 829 of the visualization system.

In comparing the initial image 1800a (shown in FIG. 18a) and the subsequent image 1800c (shown in FIG. 18c) it is noted that the respective images have the same or substantially the same focus characteristic and field of view. For example, the images inside of major sub-box 1804c and minor sub-box 1806c of FIG. 18c are consistent with the images inside the major sub-box 1804a and minor sub-box 1806a of FIG. 18a. Furthermore, the position of the treatment overlay 1810c of FIG. 18c is consistent with the position of the treatment overlay 1810a of FIG. 18a.

While the foregoing comparisons are between the initial image 1800a (shown in FIG. 18a) and each of the “shallow” subsequent image 1800b (shown in FIG. 18b) and the “deep” subsequent image 1800c (shown in FIG. 18c), it is noted that the “shallow” subsequent image 1800b and the “deep” subsequent image 1800c also have consistent field of view and focus. In other words, the images inside of major sub-box 1804c and minor sub-box 1806c of FIG. 18c are consistent with the images inside the major sub-box 1804b and minor sub-box 1806c of FIG. 18b. Furthermore, the position of the treatment overlay 1810c of FIG. 18c is consistent with the position of the treatment overlay 1810b of FIG. 18b. It is further noted that the integrated surgical system 1000 disclosed herein is configured to provide images with consistent field of view and focus, and a fixed overlay, at all depth positions between the shallow position and the deep position.

By comparison, in a surgical system that is not configured in accordance with embodiments disclosed herein (e.g., does not have a visualization system with a long depth of field and is not configured to keep or maintain the depth of field of its visualization system at its initial position relative to a volume of ocular tissue) subsequent images will have a shifted field of view and a different focus quality relative to the initial image. For example, with reference to FIGS. 15a, 18a and 18d, a subsequent image 1800d (shown in FIG. 18d) of the region of an irido-corneal angle that would be obtained after moving the visualization system 828 and the focusing objective 826 together to place the laser focus at a shallow depth d₂, without further independent movement of the field of view of the visualization system back to the initial position would have a shifted field of view and a different focus quality relative to the initial image 1800a (shown in FIG. 18a). The shifted field of view occurs because there is no further independent movement of the visualization system 828, that keeps or maintains the field of view of the visualization system at its initial position while the change in focus may occur due to movement of the short depth of field of the visualization system that places all or a portion of the target volume 60 outside the depth of field.

In further comparing the initial image 1800a (shown in FIG. 18a) and the subsequent image 1800d (shown in FIG. 18d) it is noted that the respective images have different focus characteristics and fields of view. For example, the field of view of the images inside the major sub-box 1804d and the minor sub-box 1806d is shifted relative to the field of view of the images inside the major sub-box 1804a and the minor sub-box 1806a of the initial image 1800a. Likewise, the focus of the images inside the major sub-box 1804d and the minor sub-box 1806d is not as sharp as the images inside the major sub-box 1804a and the minor sub-box 1806a of the initial image 1800a. Furthermore, the position of the treatment overlay 1810d of FIG. 18d is shifted relative to the position of the treatment overlay 1810a of FIG. 18a.

Similarly, with reference to FIGS. 16a, 18a, and 18e, a subsequent image 1800e (shown in FIG. 18e) of the region of an irido-corneal angle that would be obtained by moving the visualization system 828 and the focusing objective 826 together to place the laser focus at a deep depth d₅, without further independent movement of the field of view of the visualization system back to the initial position would have a shifted field of view and a different focus quality relative to the initial image 1800a (shown in FIG. 18a).

In further comparing the initial image 1800a (shown in FIG. 18a) and the subsequent image 1800e (shown in FIG. 18e) it is noted that the respective images have different focus characteristics and fields of view. For example, the field of view of the images inside the major sub-box 1804e and the minor sub-box 1806e is shifted relative to the field of view of the images inside the major sub-box 1804a and the minor sub-box 1806a of the initial image 1800a. Likewise, the focus of the images inside the major sub-box 1804e and the minor sub-box 1806e is not as sharp as the images inside the major sub-box 1804a and the minor sub-box 1806a of the initial image 1800a. Furthermore, the position of the treatment overlay 1810e of FIG. 18e is shifted relative to the position of the treatment overlay 1810a of FIG. 18a.

Surgical Workflow

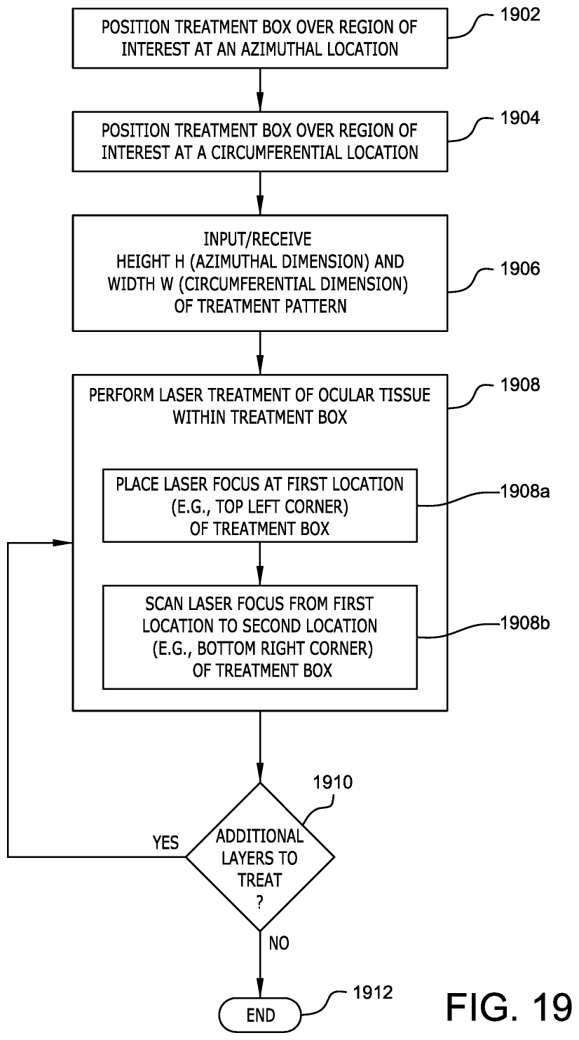

FIG. 19 is a flowchart of the workflow of surgical steps to complete a laser cut in a region of interest. The workflow is enabled by the integrated surgical system 1000 disclosed herein and may be performed by the integrated surgical system based on input received from a surgeon. As part of the workflow and with reference to FIG. 18a, a treatment box 1810a may be moved around by the user through the control system 100 of the integrated surgical system 1000 to place it over the region of interest, e.g., the trabecular meshwork. As described further below, the location of the treatment box 1810a may change due to movement of an azimuthal slider or movement of a circumferential slider. Also, as described above, the location of the treatment box 1810a may also shift due to movement of the focusing objective 826 during laser scanning.

At block 1902, and with additional reference to FIG. 18a, a treatment box 1810a is positioned over a region of interest at an azimuthal location through a graphical user interface (GUI). To this end, the vertical scale 1802 may correspond to a GUI in the form of a vertical box with a vertical slider 1811 that is configured to move up and down in response to a user activation means, e.g., mouse pointer, keyboard up/down button, etc. The numerical values at the top and bottom of the vertical scale 1802 represent the azimuthal dimension of the treatable area of the surgical system, where the treatable area is between the parallel horizontal lines 1813a, 1813b. The center of the vertical scale 1802 is taken to be zero. Thus, placing the vertical slider 1811 at zero places the vertical center of treatment box 1810a at the azimuthal center of the treatable area. As the vertical slider 1811 is moved up and down, the value of the slider changes, and the treatment box 1810a azimuthal position moves in real time. Once the treatment box 1810a is at the desired azimuthal location, the value of the vertical slider 1811 is returned to system software and taken as parameter dA.

At block 1904, and with continued reference to FIG. 18a, the treatment box 1810a is positioned over the region of interest at a circumferential location through a graphical user interface (GUI). To this end, the horizontal scale 1808 may correspond to a GUI in the form of a horizontal box with a horizontal slider 1815 that is configured to move side-to-side in response to a user activation means, e.g., mouse pointer, keyboard up/down button, etc. The numerical values at the ends of the horizontal scale 1808 represent the circumferential dimension of the treatable area of the surgical system, where the treatable area is along the length of the parallel horizontal lines 1813a, 1813b. The center of the horizontal scale 1808 is taken to be zero. Thus, placing the horizontal slider 1815 at zero places the horizontal center of the treatment box 1810 at the circumferential center of the treatable area. As the horizontal slider 1815 is moved side-to-side, the value of the slider changes, and the treatment box 1810a circumferential position moves in real time. Once the treatment box 1810a is at the desired circumferential location, the value of the horizontal slider 1815 is returned to system software and taken as parameter dC.

At block 1906, a user interface, e.g., keyboard, of the control system 100 receives inputs corresponding to the height H (azimuthal dimension) and width W (circumferential dimension) of a 3D treatment pattern through which the surgical system will scan a laser. These parameters of the treatment pattern specify a two-dimensional plane (e.g., x-y plane) referred to as a 2D channel size. Upon receipt of these inputs, the software of the control system 100 resizes the azimuthal and circumferential dimensions of the treatment box 1810a displayed on the screen, while maintaining the location of the center of the box at its location set by the positioning of the vertical slider 1811 in block 1902 and the horizontal slider 1815 set respectively in block 1904. The boundaries of the treatment box 1810a as displayed define the azimuthal and circumferential dimensions of the 3D treatment pattern.

At block 1908, a user interface, e.g., push button, of the control system 100 is activated and in response to such activation, the control system 100 performs laser treatment of the ocular tissue within the treatment box 1810a. To this end, at block 1908a and with reference to FIG. 18a, the laser focus is placed at a depth location of the 3D treatment pattern and then at a starting location of the treatment box 1810a. For example, the laser focus may be placed at the top left corner 1817 of the treatment box 1810a. The top left corner 1817 of the treatment box 1810a has coordinates (dA+H/2, dC−W/2), where:
dA=the value of the vertical slider 1811 obtained in block 1902
dC=the value of the horizontal slider 1815 obtained in block 1904
H=height/azimuthal dimension obtained in block 1908
W=width/circumferential dimension obtained in block 1908

Regarding laser focus placement, the integrated surgical system 1000 includes two galvos—one for moving the laser beam in the azimuthal direction and the other for moving the laser beam in the circumferential direction. The galvos move when voltage is applied to them. Each galvo has an empirically determined calibration constant in units of micron/V. For example, a calibration of 700 um/V for azimuthal would mean that for a change of one volt, the laser beam would move 700 um along the azimuthal axis. Each galvo has a “zero” position, which corresponds to the center of its respective scale 1802, 1808. More specifically and with reference to FIG. 18a, the zero position of the azimuthal galvo corresponds to the zero position of the vertical scale 1802, while the zero position of the circumferential galvo corresponds to the zero position of the horizontal scale 1808. From a control standpoint, this “zero” position corresponds to a certain angle that the galvo sits at about which it deviates when the laser is scanned.

Thus, as part of placing the laser focus at the starting location in block 1908a, the control system 100 uses the azimuthal and circumferential calibration constants to calculate the required azimuthal and circumferential voltages to place the laser focus at a starting location, e.g., the top left corner 1817 of the treatment box 1810a.

At block 1908b, based upon laser spot separation settings, the laser focus is then raster scanned from the top left corner 1817 of the treatment box 1810a to an end location, e.g., the bottom right corner 1819, of the treatment box. The bottom right corner 1819 of the treatment box 1810a has coordinates (dA−H/2, dC+W/2).

At block 1910, the control system 100 determines whether additional layers of the 3D treatment pattern remain to be treated. If additional layers remain, the workflow returns to block 1908, where laser treatment is performed at another depth location of the 3D treatment pattern. If no additional layers remain, the workflow ends at block 1912.

As describe above with reference to FIG. 17, during laser scanning in accordance with embodiments disclosed herein, the field of view of the visualization system remains fixed relative to the target volume 60 within the region of interest despite movement of the focusing objective during laser scanning. Because the field of view of the visualization system is fixed, the image on the display screen remains fixed, as shown in FIG. 18a-18c. Therefore, the treatment box 1810a on the display screen remains fixed in place on the screen during laser scanning.

Control System

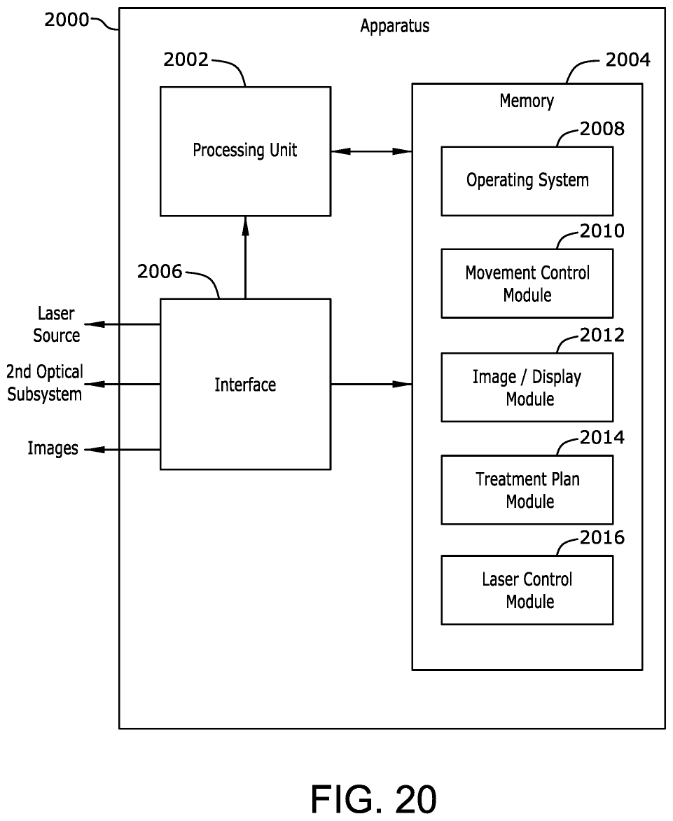

FIG. 20 is a schematic block diagram of an apparatus 2000 corresponding to the control system 100 of FIG. 8b. The apparatus 2000 is configured to execute instructions related to the imaging processes described above with reference to FIG. 17. The apparatus 2000 may be embodied in any number of processor-driven devices, including, but not limited to, a server computer, a personal computer, one or more networked computing devices, a microcontroller, and/or any other processor-based device and/or combination of devices.

The apparatus 2000 may include one or more processing units 2002 configured to access and execute computer-executable instructions stored in at least one memory 2004. The processing unit 2002 may be implemented as appropriate in hardware, software, firmware, or combinations thereof. A hardware implementation may be a general-purpose processor, a digital signal processor (DSP), an application specific integrated circuit (ASIC), a microprocessor, a microcontroller, a field programmable gate array (FPGA), a System-on-a-Chip (SOC), or any other programmable logic component, discrete gate or transistor logic, discrete hardware components, or any combination thereof, or any other suitable component designed to perform the functions described herein. Software or firmware implementations of the processing unit 2002 may include computer-executable or machine-executable instructions written in any suitable programming language to perform the various functions described herein.

The memory 2004 may include, but is not limited to, random access memory (RAM), flash RAM, magnetic media storage, optical media storage, and so forth. The memory 2004 may include volatile memory configured to store information when supplied with power and/or non-volatile memory configured to store information even when not supplied with power. The memory 2004 may store various program modules, application programs, and so forth that may include computer-executable instructions that upon execution by the processing unit 2002 may cause various operations to be performed. The memory 2004 may further store a variety of data manipulated and/or generated during execution of computer-executable instructions by the processing unit 2002.

The apparatus 2000 may further include one or more interfaces 2006 that facilitate communication between the apparatus and one or more other apparatuses. For example, the interface 2006 may be configured to communicate with the laser source 200 and components of the second optical subsystem 1002. The interface 2006 is also configured to transmit images captured by components of the second optical subsystem 1002 to the display 110 of FIG. 8b. Communication may be implemented using any suitable communications standard. For example, a LAN interface may implement protocols and/or algorithms that comply with various communication standards of the Institute of Electrical and Electronics Engineers (IEEE), such as IEEE 802.11.

The memory 2004 may store various program modules, application programs, and so forth that may include computer-executable instructions that upon execution by the processing unit 2002 may cause various operations to be performed. For example, the memory 2004 may include an operating system module (O/S) 2008 that may be configured to manage hardware resources such as the interface 2006 and provide various services to operations executing on the apparatus 2000.

The memory 2004 stores operation modules such as a movement control module 2010, an image/display module 2012, a treatment plan module 2014, and a laser control module 2016. These modules may be implemented as appropriate in software or firmware that include computer-executable or machine-executable instructions that when executed by the processing unit 2002 cause various operations to be performed, such as the operations described above with reference to FIG. 17. For example, the movement control module 2010 executes instructions that cause movement of the visualization system 828 that keeps or maintains the position of the field of view 831 of the visualization system relative to the target volume 60 of ocular tissue during a movement of a focus of a laser beam to a depth location within the target volume of ocular tissue. For example, the movement control module 2010 may execute instructions that cause a first movement mechanism, e.g., first plate 844 and objective motor 845, to move the focusing objective 826 (together with the visualization system 828), and a second movement mechanism, e.g., second plate 846 and the camera motor 847, to move the visualization system 828 as a function of the movement by the first movement mechanism. The image/display module 2012 executes instructions that cause the visualization system 828 to capture and display (through a display 110) an initial image of a region of an eye and subsequent images of the region. The treatment plan module 2014 executes instructions that cause the scanner component 830 and the objective motor 845 to scan the focus of a laser through a target volume of ocular tissue in accordance with a treatment plan. The laser control module 2016 executes instructions that cause the laser source 200 to delivery optical energy sufficient to affect treatment of the ocular tissue. For example, the laser source 200 may output energy that causes photodisruption of ocular tissue.

As an alternative to software or firmware, one or more of the modules 2010, 2012, 2014, 2016 may be implemented as appropriate in hardware. A hardware implementation may be a general-purpose processor, a DSP, an ASIC, a FPGA or other programmable logic component, discrete gate or transistor logic, discrete hardware components, or any combination thereof, or any other suitable component designed to perform the functions described herein.

### SUMMARY

Thus, disclosed herein is an integrated surgical system 1000 that provides an opto-mechanical coupling of a focusing objective 826 and a visualization system 828 that ensures a fixed, stable, real-time gonioscope imaging during surgery. Specifically, during a surgical procedure the initial image remains functionally the same throughout the procedure. In other words, the object of interest, e.g., the trabecular meshwork and the surrounding tissue, stays in focus and the field of view does not shift. Regarding the fixed field of view, in terms of imaging hardware, each feature of the object of interest is imaged to the same location of the sensor of the visualization system 828 as the focusing objective 826 moves along a second optical axis 706 in a depth direction, e.g., a z direction. Because of this, the image of the trabecular meshwork and the surrounding tissue itself is “locked” in so that the image of the trabecular meshwork and the surrounding tissue captured by the visualization system 828 during the procedure does not change in a perceivable way. In other words, the gonioscope image appears fixed with a consistent focus quality and field of view.

In some embodiments, the opto-mechanical coupling of the focusing objective 826 and the visualization system 828 is accomplished by mounting the visualization system to a second plate 846 having a camera motor 847, and then mounting the second plate 846 to a first plate 844 having the focusing objective 826 mounted thereto and an objective motor 845. The objective motor 845, operating under control of the control system 100, moves the first plate 844 to thereby move the focusing objective 826 together with the second plate 846 (and the visualization system mounted to a second plate) in the Z_{A} direction. The camera motor 847, operating under control of the control system 100, moves the second plate 846 in the Z_{B} direction to thereby move the visualization system 828 relative to the first mechanical axis 850 of the focusing objective 826, without moving the first plate 844 or the focusing objective.

As noted above in describing FIG. 18a verses FIGS. 18d and 18e, without this opto-mechanical coupling, movement of the focusing objective 826 would cause the gonioscope image to change during the procedure. For example, the image would vertically “shift” as the focusing objective 826 scans the depth of the treatment pattern. This shift would result from a combination of the off-axis imaging, where the chief optical ray angle of the visualization system 828 has an azimuthal component, and from the fact that the visualization system is mechanically attached to the first plate 844 and the objective motor 845 so that the visualization system has the same motion (direction and distance) as the motion of the focusing objective 826. Other characteristics of the gonioscope image that may change include image magnification (field of view size remains the same), image quality factors such as distortion and optical aberrations, and resolution, contrast, and quality of focus.

If these imaging characteristics are not compensated for, the usability and functionality of the visualization system 828 is adversely affected. For example, the user's ability to discern anatomical features, such as the transition regions between trabecular meshwork, scleral spur and Schwalbe's line is degraded; the vertical location of the trabecular meshwork in the image shifts as the treatment proceeds making it confusing to discern; the field of view grows or shrinks with the scan, potentially vignetting important regions of the image; and finally, the position of system overlays generated on image screens are altered relative to the desired surgical zone changes, giving a misleading impression to the user where the surgery is actually taking place.

The control system 100 is further configured to control the movement of the treatment overlay 1810a, 1810b, 1810c on the gonioscope image 1800a, 1800b, 1800c so that the overlay tracks the surgical region or surgical channel as the focusing objective 826 moves the laser focus through the depth of the target volume of ocular tissue at the surgical channel. As mentioned previously, the treatment overlay 1810a, 1810b, 1810c or treatment box is a software construction, which is a rectangle representing the size of the 2D surgical channel and is not something that is captured by the camera of visualization system 828. The treatment overlay 1810a, 1810b, 1810c is overlaid onto the gonioscope image 1800a, 1800b, 1800c by the control system 100 software and the user can control the position of the treatment overlay on the gonioscope image 1800 and the size of the overlay.

In accordance with embodiments disclosed herein, the software of the control system 100 is configured to apply a movement or transfer function to the treatment overlay 1810a, 1810b, 1810c that moves the overlay in such a manner that ensure the treatment overlay appears fixed in place relative to a location of the gonioscope image 1800a, 1800b, 1800c and maintains such appearance while the focusing objective 826 is moved during laser scanning. The transfer function of the treatment overlay 1810a, 1810b, 1810c may also be dependent on the azimuthal and circumferential galvo positions during the scanning of the laser in the x and y directions, along with laser scanning in the z direction by the objective motor and gonio stepper positions. In this case, movement of the treatment overlay 1810a, 1810b, 1810c by the control system 100 depends on the objective motor 845 position and the rate of change of azimuthal surgical position (y position) with objective motor position.

Without the overlay transfer function, the image field of view would vertically shift as the focusing objective 826 moves in the z direction and the treatment overlay 1810 would no longer occupy the same position on the gonioscope image 1800. In other words, the treatment overlay would be offset. For example, in comparing the gonio scope images of FIGS. 18a and 18d note: 1) the field of view of the gonioscope image 1800d of FIG. 18d is vertically shifted upward relative to the field of view of the gonioscope image 1800a of FIG. 18a, where the shift is most notable from the shift in images inside of the respective major sub-boxes 1804a, 1804d; and 2) the treatment overlay 1810d in FIG. 18d is vertically shifted downward relative to the treatment overlay 1810a in FIG. 18a.

Considering the treatment overlay 1810a, 1810b, 1810c or treatment box further, as previously mentioned the treatment overlay physically represents the location and size of a laser cut. The resulting laser cut will have the same size and be in the same location relative to the displayed anatomy, as the treatment overlay 1810a, 1810b, 1810c on the display screen.

While the foregoing description details features of an integrated surgical system 1000 that provides consistent field of view and focus, the system also provides for consistency with respect to other visual characteristics of images, such as resolution, distortion, and magnification, of the subsequent image 1800b, 1800c is the same as or within an acceptable tolerance of those of the initial image 1800a. Quantitatively, consistency in resolution among the initial image 1800a and the subsequent image 1800b, 1800c may be evidenced through modulation transfer function (MTF) measurements, which are mathematical descriptions of contrast. Consistency in distortion among the images 1800a, 1800b, 1800c may be evidenced using standard, well known algorithms, while consistency in magnification among the images may be evidenced by imaging a ruler, measuring the length of the ruler in pixels, and then calculating its imaged length with knowledge of the detector pixel size.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of imaging a region of an eye including a target volume of ocular tissue, the method comprising:
placing a depth of field and a field of view of a visualization system at respective positions relative to the target volume of ocular tissue;
obtaining an image of the region of the eye with the visualization system;
maintaining the position of the field of view of the visualization system relative to the target volume of ocular tissue during a movement of a focus of a laser beam to a depth location within the target volume of ocular tissue; and
repeating the maintaining for each of a plurality of movements of the focus of the laser beam to different depth locations within the target volume of ocular tissue.

2. The method of claim 1, wherein the visualization system is optically coupled to a focusing objective and placing the depth of field and the field of view of the visualization system at respective positions relative to the target volume of ocular tissue so the target volume is within the depth of field and within the field of view comprises one or more of:
moving the focusing objective along a first mechanical axis; and
moving the visualization system along a second mechanical axis.

3. The method of claim 2, wherein moving the focusing objective along a first mechanical axis comprises moving the visualization system together with the focusing objective.

4. The method of claim 1, wherein the laser beam and the visualization system are optically coupled to a focusing objective and maintaining the position of the field of view of the visualization system relative to the target volume of ocular tissue during a movement of a focus of a laser beam to a depth location within the target volume of ocular tissue comprises:
moving the focusing objective along a first mechanical axis to cause the movement of the focus of the laser beam, and
moving the visualization system along a second mechanical axis.

5. The method of claim 4, wherein moving the focusing objective along a first mechanical axis comprises moving the visualization system together with the focusing objective.

6. The method of claim 4, wherein moving the visualization system along a second mechanical axis comprises moving the visualization system without moving the focusing objective.

7. The method of claim 4, wherein moving the focusing objective and moving the visualization system occur sequentially.

8. The method of claim 4, wherein moving the focusing objective and moving the visualization system occur simultaneously.

9. The method of claim 1, wherein the target volume of ocular tissue has a thickness, the depth of field has a center, and placing the depth of field relative to the target volume so the target volume is within the depth of field comprises positioning the center of the depth of field at or near the center of the thickness of the target volume.

10. The method of claim 9, wherein the depth of field is greater than the thickness of the target volume.

11. The method of claim 10, wherein the depth of field is in a range of 2 times to 10 times greater than the thickness of the target volume.

12. A system for imaging a region of an eye including a target volume of ocular tissue, the system comprising:
a laser source configured to output a laser beam;
a first optical subsystem configured to be coupled to the eye;
a focusing objective optically coupled with the first optical subsystem and the laser source;
a visualization system having a depth of field and a field of view, the visualization system optically coupled to the focusing objective and to the first optical subsystem through the focusing objective;
a movement subsystem configured to move the focusing objective and the visualization system; and
a control system configured to control the movement subsystem and the visualization system to:
place the depth of field and the field of view of the visualization system at respective positions relative to the target volume of ocular tissue so the target volume is within the depth of field and within the field of view,
obtain an image of the region of the eye with the visualization system, and
maintain the position of the field of view of the visualization system relative to the target volume of ocular tissue during movement of a laser focus of the laser beam to a depth location within the target volume of ocular tissue.

13. The system of claim 12, wherein the movement subsystem comprises:
a first mechanism arranged to move the focusing objective together with the visualization system, and
a second mechanism arranged to move the visualization system without moving the focusing objective.

14. The system of claim 13, wherein the control system controls the movement subsystem to maintain the position of the field of view of the visualization system by being further configured to:
control the first mechanism to move the focusing objective along a first mechanical axis together with the visualization system, and
control the second mechanism to move the visualization system along a second mechanical axis without moving the focusing objective.

15. The system of claim 14, wherein the first mechanical axis and the second mechanical axis are one of parallel each other, at an angle relative to each other, or are co-axial.

16. The system of claim 14, wherein movement by the first mechanism and movement by the second mechanism are one of sequential or simultaneous.

17. The system of claim 12, wherein the movement subsystem comprises:
a first mechanism arranged to move the focusing objective, and
a second mechanism arranged to move the visualization system.

18. The system of claim 17, wherein the control system controls the movement subsystem to maintain the position of the field of view of the visualization system by being further configured to:
control the first mechanism to move the focusing objective along a first mechanical axis, and
control the second mechanism to move the visualization system along the first mechanical axis and along a second mechanical axis.

19. The system of claim 18, wherein the first mechanical axis and the second mechanical axis are of parallel each other, at an angle relative to each other, or are co-axial.

20. The system of claim 18, wherein movement by the first mechanism and movement by the second mechanism is one of sequential or simultaneous.

21. A method of imaging a region of an eye including a target volume of ocular tissue, the method comprising:
moving a visualization system to place a field of view of the visualization system at a location relative to the region of the eye;
moving a focusing objective to place a laser focus of a laser beam at a depth location within the target volume of ocular tissue; and
moving the visualization system without moving the focusing objective to keep the field of view of the visualization system at the location relative to the region of the eye.

22. The method of claim 21, wherein moving the focusing objective to place a laser focus of a laser beam at a depth location within the target volume of ocular tissue comprises:
simultaneously moving the visualization system and the focusing objective a first distance in a first direction along a first mechanical axis.

23. The method of claim 22, wherein moving the visualization system without moving the focusing objective to keep the field of view of the visualization system at the location relative to the region of the eye comprises:
moving the visualization system a second distance in a second direction along a second mechanical axis, while maintaining the position of the focusing objective.

24. The method of claim 23, wherein the second distance is a function of the first distance.

25. The method of claim 21, wherein the visualization system is mechanically coupled to the focusing objective to move together with the focusing objective along a first mechanical axis and to move independent of the focusing objective along a second mechanical axis, and moving the visualization system to place a field of view of the visualization system at a location relative to the region of the eye comprises moving the visualization system along the second mechanical axis.

26. A system for imaging a region of an eye including a target volume of ocular tissue, the system comprising:
a laser source configured to output a laser beam;
a first optical subsystem configured to be coupled to the eye;
a second optical subsystem optically coupled to the first optical subsystem, and including: a) a focusing objective that is optically coupled to obtain the laser beam from the laser source, b) a visualization system that is optically coupled to the focusing objective, c) a first movement mechanism arranged to move the focusing objective and the visualization system, and d) a second movement mechanism arranged to move the visualization system; and
a control system coupled to the second optical subsystem and configured to control the visualization system, the first movement mechanism, and the second movement mechanism to:
move the visualization system to place a field of view of the visualization system at a location relative to the region of the eye;
move the focusing objective to place a laser focus of a laser beam at a depth location within the target volume of ocular tissue; and
move the visualization system without moving the focusing objective to keep the field of view of the visualization system at the location relative to the region of the eye.

27. The system of claim 26, wherein the control system moves the focusing objective to place the focus of the laser beam at the depth location by being configured to activate the first movement mechanism to move the focusing objective and the visualization system a first distance in a first direction along a first mechanical axis.

28. The system of claim 26, wherein the control system moves the visualization system without moving the focusing objective by being configured to activate the second movement mechanism to move the visualization system a second distance in a second direction along a second mechanical axis.

29. The system of claim 26, wherein the first movement mechanism comprises:
a first structure carrying each of the visualization system and the focusing objective; and
a first motor coupled to the first structure and configured to move the first structure along a first mechanical axis.

30. The system of claim 26, wherein the second movement mechanism comprises:
a second structure carrying the visualization system but not the focusing objective; and
a second motor coupled to the second structure and configured to move the second structure along a second mechanical axis.

31. The system of claim 30, wherein the second movement mechanism is carried by the first movement mechanism.
